(19) United States
(12) Patent Application Publication
(10) Pub. No.: US 2014/0171448 A1
(43) Pub. Date: Jun. 19, 2014

(54) USE OF NICOTINIC ACETYLCHOLINE RECEPTOR ALPHA 7 ACTIVATORS

(71) Applicants: Dominik Feuerbach, Basel (CH); Baltazar Gomez-Mancilla, Basel (CH); Cristina Lopez-Lopez, Basel (CH); Donald Johns, Cambridge, MA (US); Thérése Di Paolo, Quebec (CA)

(72) Inventors: Dominik Feuerbach, Basel (CH); Baltazar Gomez-Mancilla, Basel (CH); Cristina Lopez-Lopez, Basel (CH); Donald Johns, Cambridge, MA (US); Thérése Di Paolo, Quebec (CA)

(73) Assignee: NOVARTIS AG, Basel (CH)

(21) Appl. No.: 13/982,088

(22) PCT Filed: Jan. 20, 2012
(86) PCT No.: PCT/EP12/50893

§ 371 Date: Mar. 7, 2014

Related U.S. Application Data
(60) Provisional application No. 61/436,871, filed on Jan. 27, 2011.

(51) Int. Cl. A61K 31/506; A61K 45/06; A61K 31/444; A61K 31/439; A61K 31/198

(52) U.S. Cl. CPC A61K 31/506; A61K 31/439; A61K 31/198; A61K 31/444; A61K 45/06
USPC 514/256; 514/305; 514/567

(57) ABSTRACT

The invention concerns the use of a nicotinic acetylcholine receptor alpha 7 activators for the treatment, prevention or delay of progression of a Movement Disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

The present invention relates to pharmaceutical uses of nicotinic acetylcholine receptor alpha 7 (α7-nAChR) activators, i.e. α7-nAChR agonists or positive allosteric modulators.

Movement disorders are neurological conditions that affect the speed, fluency, quality, and ease of movement. Abnormal fluency or speed of movement (dyskinesia) may involve excessive or involuntary movement (hyperkinesia) or slowed or absent voluntary movement (hypokinesia). The treatment of Movement disorders represents a high clinical need.

Compounds described as α7-nAChR agonists or α7-nAChR positive allosteric modulators have been described in, e.g. WO2001/85727, WO2004/022556, WO2005/118535, WO2005/123732, WO2006/005608, WO2007/045478, WO2007/068476, WO2007/068475 and Haydar et al (Current Topics in Medicinal Chemistry, 2010, 10, 144-152).

It has been found that α7-nAChR agonists or α7-nAChR positive allosteric modulators may be used in the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

Accordingly, a first aspect of the invention concerns the use of a α7-nAChR agonist or a α7-nAChR positive allosteric modulator for the treatment (whether therapeutic or prophylactic), prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

One embodiment of said first aspect concerns the use of a α7-nAChR agonist for the treatment (whether therapeutic or prophylactic), prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

Another embodiment of said first aspect concerns the use of a α7-nAChR positive allosteric modulator for the treatment (whether therapeutic or prophylactic), prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

A further aspect of the invention relates to a method for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonismin a subject in need of such treatment, which comprises administering to said subject a therapeutically effective amount of a α7-nAChR agonist or a α7-nAChR positive allosteric modulator.

One embodiment of said further aspect relates to a method for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism in a subject in need of such treatment, which comprises administering to said subject a therapeutically effective amount of a α7-nAChR agonist.

Another embodiment of said further aspect relates to a method for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism in a subject in need of such treatment, which comprises administering to said subject a therapeutically effective amount of a α7-nAChR positive allosteric modulator.

A further aspect of the invention relates to a method for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism in a subject in need of such treatment, which comprises (i) diagnosing said movement disorder in said subject and (ii) administering to said subject a therapeutically effective amount of a α7-nAChR agonist or a α7-nAChR positive allosteric modulator.

One embodiment of said further aspect relates to a method for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism in a subject in need of such treatment, which comprises (i) diagnosing said movement disorder in said subject and (ii) administering to said subject a therapeutically effective amount of a α7-nAChR agonist. Another embodiment of said further aspect relates to a method for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism in a subject in need of such treatment, which comprises (i) diagnosing said movement disorder in said subject and (ii) administering to said subject a therapeutically effective amount of a α7-nAChR positive allosteric modulator.

A further aspect of the invention relates to a pharmaceutical composition comprising a α7-nAChR agonist or a α7-nAChR positive allosteric modulator for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

One embodiment of said further aspect relates to a pharmaceutical composition comprising a α7-nAChR agonist or a α7-nAChR positive allosteric modulator for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

Another embodiment of said further aspect relates to a pharmaceutical composition comprising a α7-nAChR agonist or a α7-nAChR positive allosteric modulator for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

A further aspect of the invention relates to the use of a α7-nAChR agonist or a α7-nAChR positive allosteric modulator for the manufacture of a medicament for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

One embodiment of said further aspect relates to the use of a α7-nAChR agonist for the manufacture of a medicament for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

Another embodiment of said further aspect relates to the use of a α7-nAChR positive allosteric modulator for the manufacture of a medicament for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

### Movement Disorders:

Unless defined otherwise herein, the term “Instant Movement Disorder” relates to a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

“Dystonia” relates to a neurologic movement disorder characterized by sustained muscle contractions that frequently cause twisting or repetitive movements and abnormal, sometimes painful, postures or positions. It may affect any part of the body and may involve any voluntary muscle in the body.

“Dyskinesia” relates to a movement disorder characterized by the difficulty or distortion in performing voluntary movements and the presence of involuntary movements, similar to tics or chorea. Dyskinesia can be anything from a slight tremor of the hands to uncontrollable movement of most commonly the upper body but can also be seen in the lower extremities. Dyskinesia can be also classified as a symptom of several medical disorders and distinguished by the underlying cause.

“Chorea” relates to a movement disorder characterized by brief, quasi-purposeful, irregular contractions that are not repetitive or rhythmic, but appear to flow from one muscle to the next. These ‘dance-like’ movements often occur with athetosis, which adds twisting and writhing movements. Chorea can occur in a variety of conditions and disorders such Huntington's disease, Ataxia telangiectasia or Wilson's disease, among others.

“Restless Legs Syndrome” (or “Wittmaack-Ekbom Syndrome”) relates to a sensory and movement disorder with a profound impact on sleep characterized by an irresistible urge to move the body to stop uncomfortable sensations. Relief with movement of the affected limb—typically the legs and, not uncommonly, the arms—is one of the distinguishing features.

“Tics” relate to involuntary movements or vocalizations that are usually of sudden onset, brief, repetitive, stereotyped but non rhythmical in character, frequently imitating normal behavior, often occurring out of a background of normal activity. Tics can be classified as motor or vocal and can also be categorized as simple or complex. Tics can be classified as transient Tics (e.g. multiple motor and/or vocal tics within a duration between four weeks and twelve months), chronic Tics (e.g. multiple motor or vocal tics being present for more than a year) and Tourette Syndrome.

“Tremors” relate to an involuntary quasi-rhythmic, muscle contraction and relaxation involving to-and-fro movements (oscillations or twitching) of one or more body parts. It is the most common of all involuntary movements and can affect the hands, arms, eyes, face, head, vocal cords, trunk, and legs. Most tremors occur in the hands. In some people, tremor is a symptom of another neurological disorder, including multiple sclerosis, stroke, traumatic brain injury, chronic kidney disease and a number of neurodegenerative diseases that damage or destroy parts of the brainstem or the cerebellum.

“Myoclonus” relates to sudden, brief, shock-like movements, which can be positive or negative. Positive myoclonus results in contraction of a muscle or multiple muscles. Asterixis, or negative myoclonus, occurs with brief momentary loss of agonist muscle tone and subsequent contraction of antagonist muscles, resulting in a flapping motion. These nonsuppressible movements often have a characteristic saw-tooth pattern and usually disappear during sleep.

“Startle” relates to a stereotypical response to a sudden and unexpected stimulus. In most instances, the stimulus is acoustic, but other modalities such as tactile, visual, or vestibular are also effective stimuli. Exaggerated startle, is a feature of various neurologic and psychiatric conditions. Hyperekplexia is an uncommon clinical syndrome that is characterized by brisk and generalized startle in response to trivial (most often acoustic or tactile) stimulation.

“Stiff Person Syndrome” (e.g. Moersch-Woltman Condition) relates to a rare neurologic disorder of unknown etiology characterized by involuntary painful spasms and rigidity of muscles, usually involving the lower back and legs. Sub-variants include Stiff Baby Syndrome and Stiff Limb Syndrome. Prognosis is variable and there is no reliable predictor of speed and severity of disease onset. Muscle tetany may lead to muscle rupture and broken bones, or problems swallowing and breathing in severe cases

“Gait Disorders” relate to an abnormality in the manner or style of walking, which usually results from neuromuscular, arthritic, or other body changes. Gait disorders can be classified according to the system responsible for the abnormal locomotion, according to the underlying disease associated with the abnormal gait or by its phenomenology. Parkinsonian gait disturbances may also be sub-classified as continuous (appearing whenever the patient walks) and episodic (lasting seconds).

In one embodiment, the Instant Movement Disorder is a movement disorder selected from Dystonia, Dyskinesia, Chorea, Restless Legs Syndrome, Tics, Tremor, Myoclonus, Startle, Stiff Person Syndrome and Gait Disorder.

In one embodiment, the Instant Movement Disorder is Dystonia.

In one embodiment, the Instant Movement Disorder is Dyskinesia.

In one embodiment, the Instant Movement Disorder is Chorea.

In one embodiment, the Instant Movement Disorder is Chorea with the exception of Chorea in Huntington's Disease.

In one embodiment, the Instant Movement Disorder is Restless Legs Syndrome.

In one embodiment, the Instant Movement Disorder is Tics.

In one embodiment, the Instant Movement Disorder is Simple Tics.

In one embodiment, the Instant Movement Disorder is Complex Tics.

In one embodiment, the Instant Movement Disorder is Complex Tics with the exception of Tourette Syndrome.

In one embodiment, the Instant Movement Disorder is transient Tics.

In one embodiment, the Instant Movement Disorder is chronic Tics.

In one embodiment, the Instant Movement Disorder is Tremor.

In one embodiment, the Instant Movement Disorder is Myoclonus.

In one embodiment, the Instant Movement Disorder is Startle.

In one embodiment, the Instant Movement Disorder is Stiff Person Syndrome.

In one embodiment, the Instant Movement Disorder is Gait Disorder.

“Parkinson's Disease” relates to Primary Parkinsonism (isolated Parkinsonism due to a neurodegenerative process without any secondary systemic cause). Clinically, it is characterized by bradykinesia, tremor at rest, and muscle rigidity, as well as a host of other motor and non motor signs. Onset is typically during the sixth or seventh decade, with a slowly progressive course. Pathologically, the motor signs are due to gradual loss of dopaminergic cells, primarily in the substantia nigra, but neurons throughout the brain are affected, both dopaminergic and nondopaminergic.

“Symptomatic Parkinsonism” relates to conditions which feature clinical manifestations resembling Primary Parkinsonism. Symptomatic Parkinsonism includes, but is not limited to, Postencephalitic Parkinsonism (e.g. caused by viral illness triggering degeneration of nerve cells in substantia nigra), Arteriosclerotic Parkinsonism (caused by damages to brain vessels due to multiple small strokes), Drug-induced Parkinsonism (e.g. antipsychotics, metoclopramide), Parkinsonism caused by Diffuse Lewy Body Disorder (disorder characterized by the presence of Lewy bodies-clumps of alpha-synuclein and ubiquitin protein in neurons), Parkinsonism caused by Multiple System Atrophy (neurodegenerative disorder associated with the degeneration of nerve cells in specific areas of the brain, e.g. Parkinsonism caused by Striatonigral Degeneration) and Parkinsonism caused by Cortico Basal Ganglionic Degeneration (a progressive neurodegenerative disease involving the cerebral cortex and the basal ganglia).

Studies relating to the effect of nicotine and/or nAChR-modulators in patients/models of Parkinson's Disease and/or Symptomatic Parkinsonism are described in Campos et al, Neurochemistry International, 56, 2010, 850-855; Kulak et al, Brain Research, 999, 2004, 193-202; Chen et al, Neurology, 74, 2010, 878-884; Quik et al, Biochemical Pharmacology, 74, 2007, 1224-1234; and Quik et al, Ann Neurol, 62, 2007, 588-596.

In one embodiment, the Instant Movement Disorder is Parkinson's Disease.

In one embodiment, the Instant Movement Disorder is Symptomatic Parkinsonism.

In one embodiment, the Instant Movement Disorder is Symptomatic Parkinsonism selected from Postencephalitic Parkinsonism, Arteriosclerotic Parkinsonism, Drug-induced Parkinsonism, Parkinsonism caused by Diffuse Lewy Body Disorder, Parkinsonism caused by Multiple System Atrophy and Parkinsonism caused by Cortico Basal Ganglionic Degeneration.

In one embodiment, the Instant Movement Disorder is Postencephalitic Parkinsonism.

In one embodiment, the Instant Movement Disorder is Arteriosclerotic Parkinsonism.

In one embodiment, the Instant Movement Disorder is Drug-induced Parkinsonism.

In one embodiment, the Instant Movement Disorder is Parkinsonism caused by Diffuse Lewy Body Disorder.

In one embodiment, the Instant Movement Disorder is Parkinsonism caused by Multiple System Atrophy.

In one embodiment, the Instant Movement Disorder is Parkinsonism caused by Striatonigral Degeneration.

In one embodiment, the Instant Movement Disorder is Parkinsonism caused by Cortico Basal Ganglionic Degeneration.

Movement Disorders—Dyskinesia being Dyskinesia Associated with Dopamine Agonist Therapy in Symptomatic Parkinsonism:

The most commonly used treatment for Parkinson's Disease and/or Symptomatic Parkinsonism is dopamine agonist therapy, for example by administration of L-dopa (levodopa) in combination with a decarboxylase inhibitor (e.g. carbidopa). However, for many patients, a long term dopamine agonist therapy causes involuntary movements (dyskinesias) as a significant side effect (for review: Fabbrini et al, Movement Disorders, 2007, 22(10), 1379-1389; Konitsiotis, Expert Opin Investig Drugs, 2005, 14(4), 377-392; Brown et al, IDrugs, 2002, 5(5), 454-468). Consequently, there is a need for effective regimes for inhibiting or treating dyskinesia, which can be carried out without adversely affecting anti-Parkinson's Disease treatments or anti-Symptomatic Parkinsonism treatments.

α7-nAChR agonists or α7-nAChR positive allosteric modulators may be used in the treatment, prevention or delay of progression of dyskinesia associated with dopamine agonist therapy in Symptomatic Parkinsonism. α7-nAChR agonists or α7-nAChR positive allosteric modulators may be used in the treatment, prevention or delay of progression of said dyskinesia, wherein the therapy comprises the administration of levodopa.

Accordingly, in one embodiment of the invention, the Dyskinesia is dyskinesia associated with dopamine agonist therapy in Symptomatic Parkinsonism.

The term “dopamine agonist therapy” as used herein, unless indicated otherwise, means any therapy that increases dopamine receptor stimulation, including, but not limited to, therapies that directly stimulate dopamine receptors (such as administration of bromocriptine) and therapies that increase the levels of dopamine (such as administration of levodopa or of drugs which inhibit dopamine metabolism).

Dopamine agonist therapies include, but are not limited to, therapies which comprise the administration of one or more of the following agents:

levodopa (or L-dopa being a precursor of dopamine);

levodopa in combination with a levodopa decarboxylase inhibitor, such as carbidopa or benserazide;

levodopa in combination with a catechol-O-methyl transferase inhibitor, such as tolcapone or entacapone;

a monoamine oxidase B-inhibitor, such as selegiline or rasagiline;

a dopamine receptor agonist, such as bromocriptine, pergolide, pramipexole, ropinirole, cabergoline, apomorphine or lisuride.

The term “dopamine agonist” as used herein, unless otherwise indicated, means any agent that increases dopamine receptor stimulation. Preferred dopamine agonists are selected from levodopa; levodopa in combination with a levodopa decarboxylase inhibitor; levodopa in combination with a catechol-O-methyl transferase inhibitor; a monoamine oxidase B-inhibitor and a dopamine receptor agonist.

In one embodiment of the invention, the therapy comprises the administration of levodopa. Due to prevalence of associated dyskinesia, the daily dosage of levodopa for an effective dopamine agonist therapy of Symptomatic Parkinsonism needs to be determined for each patient individually and ranges typically from 250 to 1500 mg. Said total daily dose is distributed between 2-6 administrations per day, e.g. 3-6 administrations of 50-100 mg per administration. Usually, the daily dosage of levodopa needed for an effective therapy increases during the course of the therapy.

In one embodiment of the invention, the therapy comprises the administration of levodopa in combination with a levodopa decarboxylase inhibitor, such as carbidopa or benserazide.

The term “dyskinesia associated with dopamine agonist therapy”, as used herein, unless otherwise indicated, means any dyskinesia which accompanies, or follows in the course of, dopamine agonist therapy, or which is caused by, related to, or exacerbated by dopamine agonist therapy, wherein dyskinesia and dopamine agonist therapy are as defined above. Such dyskinesia often, although not exclusively, occurs as a side-effect of said dopamine agonist therapies of Symptomatic Parkinsonism.

Characteristics of such dyskinesias include motor impairment, e.g. the appearance of slow and uncoordinated involuntary movements, shaking, stiffness and problems walking.

For example, patients treated with levodopa often have reduced symptoms of Symptomatic Parkinsonism but they experience increasing difficulties to remain standing or even sitting. After prolonged use of levodopa, a majority of patients develop such dyskinesia. Dyskinesia can occur at any time during the cycle of treatment with levodopa.

In one embodiment, the α7-nAChR agonists or α7-nAChR positive allosteric modulators are for the treatment of dyskinesia, wherein the therapy comprises administration of levodopa, and said dyskinesia occurs at the time of peak levodopa plasma concentrations in the patient.

In one embodiment, the α7-nAChR agonists or α7-nAChR positive allosteric modulators are for the treatment of dyskinesia, wherein the therapy comprises administration of levodopa, and said dyskinesia occurs when the levodopa plasma concentrations in a patient rise or fall (diphasic dyskinesia).

One aspect of the treatment of dyskinesias associated with dopamine agonist therapy in Symptomatic Parkinsonism is that said treatment should have a minimal adverse effect on the treatment of Symptomatic Parkinsonism itself, which is effected by the dopamine agonist therapy. For example: neuroleptics, which can be used to treat dyskinesias, have an adverse effect on the efficiency of the dopamine agonist therapy, for example in parameters associated with cognition, depression and sleep behavior of Symptomatic Parkinsonism patients. Highly relevant would be an anti-dyskinetic agent that has a positive effect on the treatment of Symptomatic Parkinsonism itself, e.g. improving parameters associated with cognition.

Movement Disorders—Treatment of Symptomatic Parkinsonism with a Combination of a Dopamine Agonist and an α7-nAChR Agonist and/or Positive Allosteric Modulator:

Surprisingly it was found that α7-nAChR agonists and/or positive allosteric modulators are able to prolong the action of dopamine agonists, e.g. levodopa, in the treatment of Parkinson's Disease and/or Symptomatic Parkinsonism. Consequently, compared to therapies using such dopamine agonists, the time interval for administration of said dopamine agonists may be prolonged leading to a lower daily dosage needed to achieve equal control of Parkinson's Disease and/or Symptomatic Parkinsonism.

A further aspect of the invention relates to a method for the treatment or delay of progression of Symptomatic Parkinsonism in a subject in need of such treatment, which comprises

administering to said subject a therapeutically effective amount of (i) a dopamine agonist and (ii) a α7-nAChR agonist or a α7-nAChR positive allosteric modulator,
wherein the daily dosage of the dopamine agonist is reduced compared to the daily dosage of said dopamine agonist needed to reach an equal control of Symptomatic Parkinsonism in the subject without co-administration of the α7-nAChR agonist or the α7-nAChR positive allosteric modulator.

In a preferred embodiment, said dopamine agonist comprises levodopa.

In a further preferred embodiment, said reduced daily dosage is a dosage reduced by at least 10%.

In a further preferred embodiment, said reduced daily dosage is a dosage reduced by at least 20%.

In a further preferred embodiment, said reduced daily dosage is achieved by administering the dopamine agonist in larger time intervals.

### General Aspects in the Treatment of Movement Disorders:

Treatment may comprise a reduction in the characteristics associated with the Instant Movement Disorder, including e.g., although not limited to, a reduction in the scale of involuntary movements, a reduction in the number of involuntary movements, an improvement in the ability to carry out normal tasks, an improved ability to walk, increased period of time between episodes of the Instant Movement Disorder.

In the case of prophylactic treatment, the α7-nAChR agonists or α7-nAChR positive allosteric modulators may be used to delay or prevent the onset of the Instant Movement Disorder.

The term “subject” as used herein refers preferably to a human being, especially to a patient being diagnosed with the Instant Movement Disorder.

The term “therapeutically effective amount” as used herein typically refers to a drug amount which, when administered to a subject, is sufficient to provide a therapeutic benefit, e.g. is sufficient for treating, preventing or delaying the progression of the Instant movement disorder (e.g. the amount provides an amelioration of symptoms, e.g. it leads to a reduction in the scale of involuntary movements).

For the above-mentioned indications (the conditions and disorders) the appropriate dosage will vary depending upon, for example, the compound employed, the host, the mode of administration and the nature and severity of the condition being treated. However, in general, satisfactory results in animals are indicated to be obtained at a daily dosage of from about 0.01 to about 100 mg/kg body weight, preferably from about 0.1 to about 10 mg/kg body weight, e.g. 1 mg/kg. In larger mammals, for example humans, an indicated daily dosage is in the range from about 0.1 to about 1000 mg, preferably from about 1 to about 400 mg, most preferably from about 3 to about 100 mg of a α7-nAChR agonist or a α7-nAChR positive allosteric modulator conveniently administered, for example, in divided doses up to four times a day.

### Nicotinic Acetylcholine Receptor Alpha δ Agonist:

As used herein a “α7-nAChR agonist” is a compound that binds to a receptor comprising a α7-nAChR subunit in vivo and in vitro and is activating the receptor to perform its physiological function. Activation can be measured by the method disclosed in WO2001/85727, i.e. a functional affinity assay at the homomeric alpha 7 nicotinic acetylcholine receptor (α7 nAChR) carried out with a rat pituitary cell line stably expressing the α7 nAChR. As read out, the calcium influx upon stimulation of the receptor compared to epibatidine is used. “α7-nAChR agonists” according to the invention typically induce calcium influx of at least 50% of the maximal influx evoked by epibatidine with an EC₅₀ value of at least 1 μM; preferred agonists induce calcium influx of at least 75% of the maximal influx evoked by epibatidine with an EC₅₀ value of at least 400 nM; more preferred agonists induce calcium influx of at least 85% of the maximal influx evoked by epibatidine with an EC₅₀ value of at least 50 nM.

In particular, preferred α7-nAChR agonists should be well absorbed from the gastrointestinal tract, should be sufficiently metabolically stable and possess favorable pharmacokinetic properties.

Further preferred α7-nAChR agonists bind in-vivo potently to α7-nAChRs whilst showing little affinity for other receptors, especially for other nAChRs, e.g. α4β2 nAChR, for muscarinic acetylcholine receptors, e.g. M1, and/or the 5-HT₃ receptor.

Further preferred α7-nAChR agonists cross the blood brain barrier effectively.

Preferred α7-nAChR agonists should be non-toxic and demonstrate few side-effects.

Furthermore, a preferred α7-nAChR agonist will be able to exist in a physical form that is stable, non-hygroscopic and easily formulated.

In one embodiment, the α7-nAChR agonist is selective for a receptor comprising a α7-nAChR subunit, since such an agonist would be expected to cause fewer side effects than a non-selective agonist to a treated subject. An agonist being selective for a receptor comprising a α7-nAChR subunit has a functional affinity to such a receptor to a much higher degree, e.g. at least 10-fold affinity difference in ECs value, preferably at least 20-fold, more preferably at least 50-fold, compared to any other nicotinic acetylcholine receptor. To assess the affinity of the α7-nAChR agonists of the invention on other nicotinic acetylcholine receptors, the method disclosed in WO2001/85727 can be used, i.e. to assess the affinity on human neuronal α4β2 nAChR, a similar functional assay is carried out using a human embryonic kidney cell line stable expressing the human α4β2 subtype and to assess the activity of the compounds of the invention on the “ganglionic subtype” and the “muscle type” of nicotinic receptor, similar functional assays are carried out with a human embryonic kidney cell line stably expressing the human “ganglionic subtype” or a cell line endogenously expressing the human “muscle type” of nicotinic receptors.

In the last 15 years much effort has been focused on developing selective α7 nAChR agonists leading to the discovery of many different chemotypes displaying said selective activity. These efforts are summarized the review from Horenstein et al (Mol Pharmacol, 2008, 74, 1496-1511, which describes no less than 9 different families of α7 nAChR agonists, in most of which selective agonists have been found. All compounds disclosed in FIG. 1 of said review are incorporated herein by reference. In fact, several drug candidates having an α7 nAChR agonist mode of action entered pre-clinical or even clinical testing (for review: Broad et al, Drugs of the Future, 2007, 32(2), 161-170; Romanelli et al, Expert Opin Ther Patents, 2007, 17(11), 1365-1377). Examples of such compounds—again belonging to a diversity of chemotypes—are MEM3454, MEM63908, SSR180711, GTS21, EVP6124, ABT107, ABT126, TC-5619, AZD-6319 and SAR-130479. Further α7 nAChR agonists and their use as pharmaceuticals are known, for example, from WO2001/85727, WO2004/022556, WO2005/118535, WO2005/123732, WO2006/005608, WO2007/045478, WO2007/068476 and WO2007/068475.

In one embodiment, the α7-nAChR agonist is a low molecular weight compound.

In one embodiment, the α7-nAChR agonist has a maximum molecular weight of 1500 daltons.

In one embodiment, the α7-nAChR agonist has a maximum molecular weight of 1000 daltons.

In one embodiment, the α7-nAChR agonist has a maximum molecular weight of 800 daltons.

In one embodiment, the α7-nAChR agonist has a maximum molecular weight of 500 daltons.

In one embodiment, the α7-nAChR agonist is a compound of formula (I)

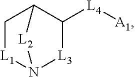

wherein

### L₁ is —CH₂—; L₂ is —CH₂— or —CH₂—CH₂—; and L₃ is —CH₂— or —CH(CH₃)—; or

### L₁ is —CH—CH₂—; L₂ is —CH₂; and L₃ is —CH₂—CH₂—;

L₄ is a group selected from

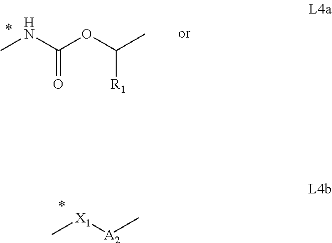

wherein the bond marked with the asterisk is attached to the azabicycloalkyl moiety;
R₁ is hydrogen or C₁₋₄ alkyl;

### X₁ is —O— or —NH—;

A₂ is selected from

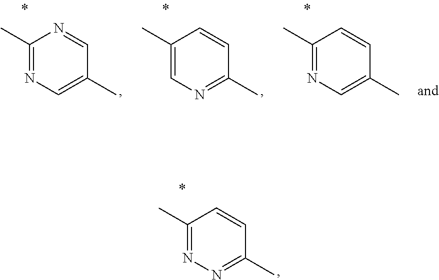

wherein the bond marked with the asterisk is attached to X₁;
A₁ is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₂, and wherein a substituent on a nitrogen in a heterocyclic ring system may not be halogen;
each R₂ independently is C₁₋₆alkyl, C₁₋₆-halogenalkyl, C₁₋₆-alkoxy, C₁₋₆halogenalkoxy, halogen, cyano or a three- to six-membered monocyclic ring system which may be aromatic, saturated or partially saturated and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₂ at adjacent ring atoms form a C₃₋₄alkylene group, wherein 1-2 carbon atoms may be replaced by X₂, and wherein the C₃alkylene group may be substituted once or more than once by R₃;
each X₂ independently is —O— or —N(R₄)—;
each R₄ independently is hydrogen or C₁₋₆alkyl; and
each R₃ independently is halogen or C₁₋₆alkyl;
in free base form or in acid addition salt form.

In one embodiment, the α7-nAChR agonist is a compound of formula (II)

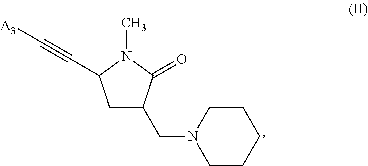

wherein
A₃ is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₅, and wherein a substituent on a nitrogen in a heterocyclic ring system may not be halogen;
each R₅ independently is C₁₋₆alkyl, C₁₋₆halogenalkyl, C₁₋₆alkoxy, C₁₋₆halogenalkoxy, halogen, cyano, amino or a three- to six-membered monocyclic ring system which may be aromatic, saturated or partially saturated and which may contain from 1 to 4 hetero atoms selected from nitrogen, oxygen and sulfur, and wherein each ring system may contain not more than 2 oxygen atoms and not more than 2 sulfur atoms, and wherein each ring system may in turn be substituted once or more than once by C₁₋₆alkyl, C₁₋₆halogenalkyl, C₁₋₆alkoxy, C₁₋₆halogenalkoxy, halogen or cyano, and wherein a substituent on a nitrogen in a heterocyclic ring system may not be halogen;
or two R₅ at adjacent ring atoms form a C₃₋₄alkylene group, wherein 1-2 carbon atoms may be replaced by X₃, and wherein the C₃₋₄alkylene group may be substituted once or more than once by R₆;
each X₃ independently is —O— or —N(R₇)—;
each R₇ independently is hydrogen or C₁₋₆alkyl; and
each R₆ independently is halogen or C₁₋₆alkyl;
in free base form or in acid addition salt form.

Unless indicated otherwise, the expressions used in this invention have the following meaning:

“Alkyl” represents a straight-chain or branched-chain alkyl group, for example, methyl, ethyl, n- or iso-propyl, n-, iso-, sec- or tert-butyl, n-pentyl, n-hexyl; C₁₋₆alkyl preferably represents a straight-chain or branched-chain C₁₋₄alkyl with particular preference given to methyl, ethyl, n-propyl, iso-propyl and tert-butyl.

Each alkyl part of “alkoxy”, “halogenalkyl” and so on shall have the same meaning as described in the above-mentioned definition of “alkyl”, especially regarding linearity and preferential size.

A substituent being substituted “once or more than once”, for example as defined for A₁, is preferably substituted by one to three substituents.

Halogen is generally fluorine, chlorine, bromine or iodine; preferably fluorine, chlorine or bromine. Halogenalkyl groups preferably have a chain length of 1 to 4 carbon atoms and are, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,2-trichloroethyl, 1,1,2,2-tetrafluoroethyl, 2,2,3,3-tetrafluoropropyl, 2,2,3,3,3-pentafluoropropyl or 2,2,3,4,4,4-hexafluorobutyl; preferably —CF₃, —CHF₂, —CH₂F, —CHF—CH₃, —CF₂CH₃, or —CH₂CF₃.

In the context of the invention, the definitions of “two R₂ at adjacent ring atoms form a C₃₋₄alkylene group, wherein 1-2 carbon atoms may be replaced by X₂” or “two R₅ at adjacent ring atoms form a C₃₋₄alkylene group, wherein 1-2 carbon atoms may be replaced by X₃” encompass —CH₂—CH₂—CH₂—, —CH₂—CH₂—CH₂—CH₂—, —O—CH₂—O—, —O—CH₂—CH₂—O— and —CH₂—CH₂—NH—. An example of a substituted group is —CH₂—CH₂—N(CH₃)—.

In the context of the invention, the definition of A₁ or A₃ as a “five- to ten-membered monocyclic or fused polycyclic aromatic ring system” encompasses a C₆- or C₁₀-aromatic hydrocarbon group or a five- to ten-membered heterocyclic aromatic ring system. “Polycyclic” means preferably bicyclic.

In the context of the invention, the definition of R₂ as a “three- to six-membered monocyclic ring system” encompasses a C₆-aromatic hydrocarbon group, a five- to six-membered heterocyclic aromatic ring system and a three- to six-membered monocyclic aliphatic or heterocyclic ring system.

A C₆- or C₁₀-aromatic hydrocarbon group is typically phenyl or naphthyl, especially phenyl.

Preferably, but also depending on substituent definition, “five- to ten-membered heterocyclic aromatic ring systems” consist of 5 to 10 ring atoms of which 1-3 ring atoms are hetero atoms. Such heterocyclic aromatic ring systems may be present as a single ring system or as bicyclic or tricyclic ring systems; preferably as single ring systems or as benz-annelated ring systems. Bicyclic or tricyclic ring systems may be formed by annelation of two or more rings, or by a bridging atom, e.g. oxygen, sulfur, nitrogen. Examples of heterocyclic ring systems are: imidazo[2,1-b]thiazole, pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazolidine, isothiazole, isothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pteridine, and the corresponding benz-annelated heterocycles, e.g. indole, isoindole, coumarin, isoquinoline, quinoline and the like. Preferred heterocycles are: imidazo[2,1-b]thiazole, oxazole, isoxazole, thiazole, isothiazole, triazole, pyrrole, furane, tetrahydrofurane, pyridine, pyrimidine, imidazole or pyrazole.

In the context of the invention, three- to six-membered monocyclic aliphatic ring systems are typically cyclopropyl, cyclobutyl, cyclopentyl or cyclohexyl.

On account of asymmetrical carbon atom(s) that may be present in the compounds of formula (I) and compounds of formula (II), the compounds may exist in optically active form or in form of mixtures of optical isomers, e.g. in form of racemic mixtures or diastereomeric mixtures. All optical isomers and their mixtures, including racemic mixtures, are part of the present invention.

In one embodiment, the α7-nAChR agonist is a compound of formula (I)

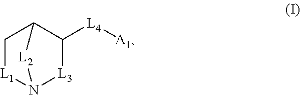

wherein

### L₁ is —CH₂—; L₂ is —CH₂—CH₂—; and L₃ is —CH₂ or —CH(CH₃)—;

L₄ is a group selected from

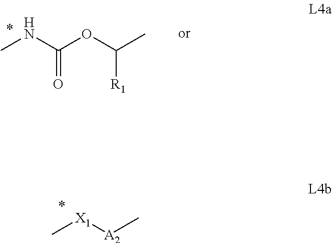

wherein the bond marked with the asterisk is attached to the azabicycloalkyl moiety;
R₁ is hydrogen or C₁₋₄alkyl;

### X₁ is —O— or —NH—;

A₂ is selected from

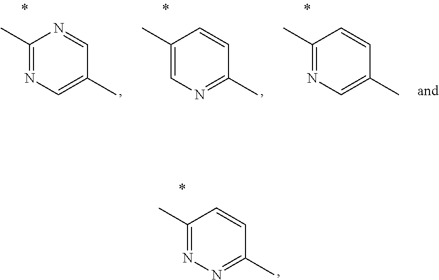

wherein the bond marked with the asterisk is attached to X₁;
A₁ is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₂, and wherein a substituent on a nitrogen in a heterocyclic ring system may not be halogen; and
each R₂ independently is C₁₋₆alkyl, C₁₋₆halogenalkyl, C₁₋₆alkoxy, C₁₋₆halogenalkoxy or halogen.

In one embodiment, the α7-nAChR agonist is a compound of formula (I) (I),

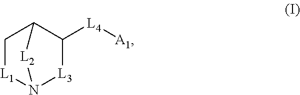

wherein

### L₁ is —CH₂—; L₂ is —CH₂—CH₂—; and L₃ is —CH₂—;

### L₄ is

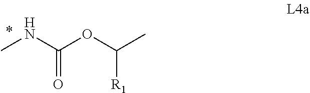

wherein the bond marked with the asterisk is attached to the azabicycloalkyl moiety;
R₁ is hydrogen or C₁₋₄alkyl;
A₁ is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₂, and wherein a substituent on a nitrogen in a heterocyclic ring system may not be halogen; and
each R₂ independently is C₁₋₆alkyl, C₁₋₆halogenalkyl, C₁₋₆alkoxy, C₁₋₆halogenalkoxy or halogen.

In one embodiment, the α7-nAChR agonist is a compound of formula (I)

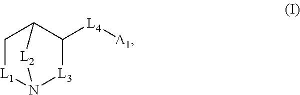

wherein

### L₁ is —CH₂—; L₂ is —CH₂CH₂—; and L₃ is —CH₂— or —CH(CH₃)—;

### L₄ is

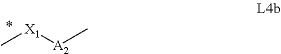

wherein the bond marked with the asterisk is attached to the azabicycloalkyl moiety;

### X₁ is —O— or —NH—;

A₂ is selected from

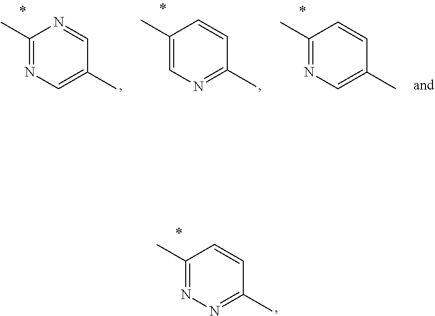

wherein the bond marked with the asterisk is attached to X₁;
A₁ is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₂, and wherein a substituent on a nitrogen in a heterocyclic ring system may not be halogen; and
each R₂ independently is C₁₋₆alkyl, C₁₋₆halogenalkyl, C₁₋₆alkoxy, C₁₋₆halogenalkoxy or halogen.

In one embodiment, the α7-nAChR agonist is a compound of formula (I)

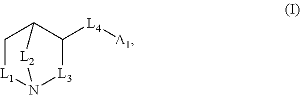

wherein

### L₁ is —CH₂—CH₂—; L₂ is —CH₂—; and L₃ is —CH₂—CH—;

### L₄ is

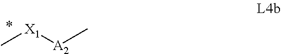

wherein the bond marked with the asterisk is attached to the azabicycloalkyl moiety;

### X₁ is —O— or —NH—;

A₂ is selected from

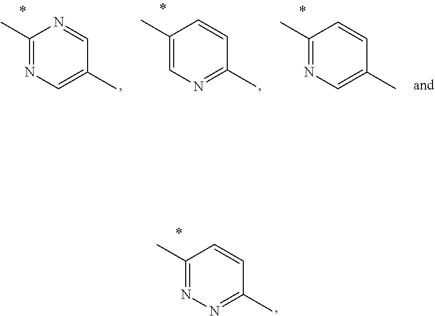

wherein the bond marked with the asterisk is attached to X₁;
A₁ is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₂, and wherein a substituent on a nitrogen in a heterocyclic ring system may not be halogen; and
each R₂ independently is C₁₋₆alkyl, C₁₋₆halogenalkyl, C₁₋₆alkoxy, C₁₋₆halogenalkoxy or halogen.

In one embodiment, the α7-nAChR agonist is a compound of formula (II)

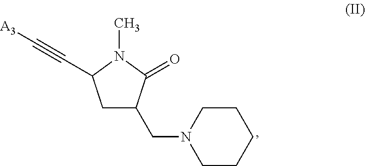

wherein
A₃ is a five- to ten-membered monocyclic or fused polycyclic aromatic ring system which may contain from 1 to 4 hetero atoms selected from nitrogen, oxygen and sulfur, wherein the ring system may contain not more than 2 oxygen atoms and not more than 2 sulfur atoms, and wherein the ring system may be substituted once or more than once by R₅, and wherein a substituent on a nitrogen in a heterocyclic ring system may not be halogen; and
each R₅ independently is C₁₋₆alkyl, C₁₋₆halogenalkyl, C₁₋₆alkoxy, C₁₋₆halogenalkoxy, amino or halogen.

In one embodiment, the α7-nAChR agonist is a compound selected from Group P1; Group P1 is the group consisting of
A-1: (S)-(1-aza-bicyclo[2.2.2]oct-3-yl)-carbamic acid (S)-1-(2-fluoro-phenyl)-ethyl ester;
A-2: (R)-(1-aza-bicyclo[2.2.2]oct-3-yl)-carbamic acid (R)-1-(2-chloro-phenyl)-ethyl ester,
A-3: (S)-(1-aza-bicyclo[2.2.2]oct-3-yl)-carbamic acid (S)-1-phenyl-ethyl ester;
B-1: (R)-3-(5-phenyl-pyrimidin-2-yloxy)-1-aza-bicyclo[2.2.2]octane;
B-2: (R)-3-(5-p-tolyl-pyrimidin-2-yloxy)-1-aza-bicyclo[2.2.2]octane;
B-3: (R)-3-(5-(2-fluoro-4-methyl-phenyl)-pyrimidin-2-yloxy)-1-aza-bicyclo[2.2.2]octane;
B-4: (R)-3-(5-(3,4-dimethyl-phenyl)-pyrimidin-2-yloxy)-1-aza-bicyclo[2.2.2]octane;
B-5: (R)-3-(6-p-tolyl-pyridin-3-yloxy)-1-aza-bicyclo[2.2.2]octane;
B-6: (R)-3-(6-phenyl-pyridin-3-yloxy)-1-aza-bicyclo[2.2.2]octane;
B-7: (R)-3-(6-(3,4-dimethyl-phenyl)-pyrindin-3-yloxy)-1-aza-bicyclo[2.2.2]octane;
B-8: (R)-3-[6-(2-fluoro-4-methyl-phenyl)-pyridazin-3-yloxy]-1-aza-bicyclo[2.2.2]octane;
B-9: (R)-3-[6-(4,5-dimethyl-2-fluoro-phenyl)-pyridazin-3-yloxy]-1-aza-bicyclo[2.2.2]octane;
B-10: (R)-3-[6-(3,4-dimethyl-phenyl)-pyridazin-3-yloxy]-1-aza-bicyclo[2.2.2]octane;
B-11: (R)-3-[6-(4-methyl-phenyl)-pyridazin-3-yloxy]-1-aza-bicyclo[2.2.2]octane;
B-12: (R)-3-[6-(2,5-difluoro-4-methyl-phenyl)-pyridazin-3-yloxy]-aza-bicyclo[2.2.2]octane;
B-13: (2S,3R)-3-[6-(1H-indol-5-yl)-pyridazin-3-yloxy]-2-methyl-1-aza-bicyclo[2.2.2]octane;
B-14: (2R,3S)-3-[6-(1H-indol-5-yl)-pyridazin-3-yloxy]-2-methyl-1-aza-bicyclo[2.2.2]octane;
B-15: (2S,3R)-3-[5-(1H-indol-5-yl)-pyrimidin-2-yloxy]-2-methyl-1-aza-bicyclo[2.2.2]octane;
B-16: (2R,3S)-3-[5-(1H-indol-5-yl)-pyrimidin-2-yloxy]-2-methyl-1-aza-bicyclo[2.2.2]octane;
B-17: 3-[6-(1H-indol-5-yl)-pyridin-3-yloxy]-2-methyl-1-aza-bicyclo[2.2.2]octane;
B-18: (2S,3R)-2-methyl-3-[6-(5-methyl-thiophen-2-yl)-pyridazin-3-yloxy]-1-aza-bicyclo[2.2.2]octane;
B-19: 3-[6-(2,3-dimethyl-1H-indol-5-yl)-pyridazin-3-yloxy]-2-methyl-1-aza-bicyclo[2.2.2]octane;
B-20: trans-2-methyl-1-aza-bicyclo[2.2.2]oct-3-yl)-(6-phenyl-pyridin-3-yl)-amine;
B-21: trans-(6-(1H-indol-5-yl)-pyridin-3-yl)-(2-methyl-1-aza-bicyclo[2.2.2]oct-3-yl)-amine;
C-1: (4S,5R)-4-[5-(1H-indol-5-yl)-pyrimidin-2-yloxy]-aza-bicyclo[3.3.1]nonane;
C-2: 5-{2-[(4S,5R)-(1-aza-bicyclo[3.3.1]non-4-yl)oxy]-pyrimidin-5-yl}-, 3-dihydro-indol-2-one;
C-3: (4S,5R)-4-[6-(1H-indol-5-yl)-pyridin-3-yloxy]-1-aza-bicyclo[3.3.1]nonane;
C-4: (4S,5R)-4-[5-(1H-indol-5-yl)-pyridin-2-yloxy]-1-aza-bicyclo[3.3.1]nonane;
C-5: (4S,5R)-4-[6-(1H-indol-5-yl)-pyridazin-3-yloxy]-1-aza-bicyclo[3.3.1]nonane;
C-6: 5-{6-[(4S,5R)-(1-aza-bicyclo[3.3.1]non-4-yl)oxy]-pyridazin-3-yl}-1,3-dihydro-indol-2-one;
C-7: (1-aza-bicyclo[3.3.1]non-4-yl)-[5-(1H-indol-5-yl)-pyridn-2-yl]-amine;
C-8: (1-aza-bicyclo[3.3.1]non-4-yl)-[5-(1H-indol-5-yl)-pyrimidin-2-yl]-amine;
C-9: (1-aza-bicyclo[3.3.1]non-4-yl)-[6-(1H-indol-5-yl)-pyridin-3-yl]-amine;
C-10: (1-aza-bicyclo[3.3.1]non-4-yl)-[6-(1H-indol-5-yl)-pyridin-3-yl]-amine;
C-11: (1-aza-bicyclo[3.3.1]non-4-yl)-[5-(1H-indol-4-yl)-pyrimidin-2-yl]-amine;
C-12: (1-aza-bicyclo[3.3.1]non-4-yl)-[6-(1H-indol-5-yl)-pyridazin-3-yl]-amine;
D-1: 5-benzofuran-5-ylethynyl-1-methyl-3-piperidin-1-ylmethyl-pyrrolidin-2-one;
D-2: 1-methyl-5-phenylethynyl-3-piperidin-1-ylmethyl-pyrrolidin-2-one;
D-3: 1-methyl-5-(1-methyl-1H-indol-5-ylethynyl)-3-piperidin-1-ylmethyl-pyrrolidin-2-one;
D-4: 5-(3-Amino-phenylethynyl)-1-methyl-3-piperidin-1-ylmethyl-pyrrolidin-2-one;
E-1: 4-(5-phenyl-1,3,4-thiadiazol-2-yloxy)-1azatricyclo[3.3.1.1^{3,7}]decane having the formula

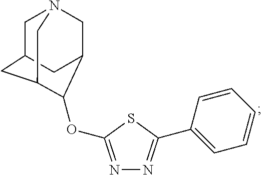 [SMILES: c1ccc(-c2nnc(OC3C4CC5CC3CN(C5)C4)s2)cc1]

E-1a: (4S)-4-(5-phenyl-1,3,4-thiadiazol-2-yloxy)-1 azatnicydlo[3.3.1.1^{3,7}]decane;
E-1b: 4-(6-(1H-indol-5-yl)-pyridazin-3-yloxy)-1 azatricyclo[3.3.1.1^{3,7}]decane;
E-1c: 4-(6-(1H-indol-5-yl)-pyridin-3-yloxy)-1 azatricyclo[3.3.1.1^{3,7}]decane;
E-1d: 4-(5-(1H-indol-5-yl)-pyrimidin-2-yloxy)-1 azatricyclo[3.3.1.1^{3,7}]decane;
E-2: 2-(6-phenylpyridazine-3-yl)octahydropyrrolo[3,4-c]pyrrole having the formula

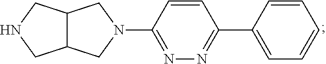 [SMILES: c1ccc(-c2ccc(N3CC4CNCC4C3)nn2)cc1]

E-3: 5-[6-(5-methyl-hexahydro-pyrrolo[3,4-c]pyrrol-2-yl-pyridazin-3-yl]H-indole having the formula

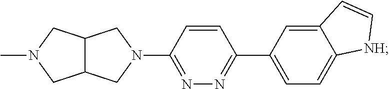 [SMILES: CN1CC2CN(c3ccc(-c4ccc5[nH]ccc5c4)nn3)CC2C1]

E-3a: 5-[6-(cis-5-methyl-hexahydro-pyrrolo[3,4-c]pyrrol-2-yl-pyridazin-3-yl1H-indole;
E-4: 5-[5-(6-methyl-3,6-diaza-bicyclo[3.2.0]hept-3-yl)-pyridin-2-yl]-1H-indole having the formula

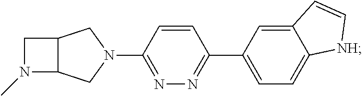 [SMILES: CN1CC2CN(c3ccc(-c4ccc5[nH]ccc5c4)nn3)CC21]

E-4-a: 5-[5-{(1R,5R)-6-methyl-3,6-diaza-bicyclo[3.2.0]hept-3-yl}-pyridin-2-yl]-1H-indole
E-5: 2-Methyl-5-(6-phenyl-pyridazin-3-yl)-octahydro-pyrrolo[3,4-c]pyrrole having the formula

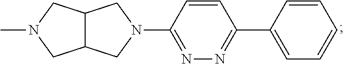 [SMILES: CN1CC2CN(c3ccc(-c4ccccc4)nn3)CC2C1]

E-6: 5-{6-[1-azabicyclo[2.2.2]oct-3-yloxy]pyridazin-3-yl}-1H-indole;
E-6a: 5-{6-[(3R)-1-azabicyclo[2.2.2]oct-3yloxy]pyridazin-3-yl}-1H-indole;
E-7: 5-{6-[(3-azabicyclo[2.2.2]oct-3-yloxy]pyridazin-3-yl}-1,3-dihydro-indol-2-one;
E-7a: 5-{6-[(3R)1-azabicyclo[2.2.2]oct-3-yloxy]pyridazin-3-yl}-1,3-dihydro-indol-2-one;
E-8: N-(1-azabicyclo[2.2.2]oct-3-yl)-1H-indazole-3-carboxamide;
E-8a: N-((3R)-1-azabicyclo[2.2.2]oct-3-yl)-1H-indazole-3-carboxamide
E-8b: N-((3S)-1-azabicyclo[2.2.2]oct-3-yl)-1H-indazole-3-carboxamide
E-9: N-(1-azabicyclo[2.2.2]oct-3-yl)-5-(trifluoromethoxy)-1H-indazole-3-carboxamide;
E-9a: N-((3R)-1-azabicyclo[2.2.2]oct-3-yl)-5-(trifluoromethoxy)-1H-indazole-3-carboxamide;
E-9b: N-((3S)-1-azabicyclo[2.2.2]oct-3-yl)-5-(trifluoromethoxy)-1H-indazole-3-carboxamide;
E-10: N-(2-((3-pyridinyl)methyl)-1-azabicyclo[2.2.2]oct-3-yl)benzofuran-2-carboxamide;
E-10a: (2S,3R)—N-(2-((3-pyridinyl)methyl)-1-azabicyclo[2.2.2]oct-3-yl)benzofuran-2-carboxamide;
E-11: N-(2-((3-pyridinyl)methyl)-1-azabicyclo[2.2.2]oct-3-yl)-3,5-difluorobenzamide;
E-11a: (2S,3R)—N-(2-((3-pyridinyl)methyl)-1-azabicyclo[2.2.2]oct-3-yl)-3,5-difluorobenzamide;
E-11b: N-(2-((3-pyridinyl)methyl)-1-azabicyclo[2.2.2]oct-3-yl)-5-methylthiophene-2-carboxamide;
E-11c: (2S,3R)—N-(2-((3-pyridinyl)methyl)-1-azabicyclo[2.2.2]oct-3-yl)-5-methylthiophene-2-carboxamide;
E-11d: N-(2-((3-pyridinyl)methyl)-1-azabicyclo[2.2.2]oct-3-yl)-5-(2-pyridinyl)thiophene-2-carboxamide;
E-11e: (2S,3R)—N-(2-((3-pyridinyl)methyl)-1-azabicyclo[2.2.2]oct-3-yl)-5-(2-pyridinyl)thiophene-2-carboxamide;
E-12: 4-(5-methyloxazolo[4,5-b]pyridin-2-yl)-1,4-diazabicyclo[3.2.2]nonane;
E-13: [N-[(3R)-1-azabicyclo[2.2.2]oct-3-yl]-4-chlorobenzamide;
E-14: furo[2,3-c]pyridine-5-carboxylic acid (1-aza-bicyclo[2.2.2]oct-3-yl)-amide;
E-15: 2,3-dihydro-benzo[1,4]dioxine-6-carboxylic acid (1-aza-bicyclo[2.2.2]oct-3-yl)-amide;
E-16: 5-morpholin-4-yl-pentanoic acid (4-pyridine-3-yl-phenyl)-amide;
E-17: N-{4-[4-(2,4-dimethoxy-phenyl)-piperazin-1-yl]-butyl}-4-pyridin-2-yl-benzamide;
E-18: 1-[6-(4-fluorophenyl)pyridin-3-yl]-3-(4-piperidin-1-ylbutyl)-urea;
E-19: 7,8,9,100-tetrahydro-6,110-methano-6H-pyrazino-(2,3-h) (3)-benzazepine;
E-20: (2′R)-spiro-[1-azabicyclo[2.2.2]octane-3,2′(3′H)-furo[2,3-b]pyridine];
E-21: 1,4-Diaza-bicyclo[3.2.2]nonane-4-carboxylic acid 4-bromo-phenyl ester;
E-22: 3-[1-(2,4-Dimethoxy-phenyl)-meth-(E)-ylidene)-3,4,5,6-tetrahydro-[2,3′]bipyridinyl;
E-23: 7-(2-Methoxy-phenyl)-benzofuran-2-carboxylic acid (1-aza-bicyclo[2.2.2]oct-3-yl)-amide;
E-24: N-methyl-1-{5-[3′H-spiro[4-azabicyclo[2.2.2]octane-2,2′-furo-[2,3-b]pyridine-5′-yl]-2-thienyl}methanamine having the formula

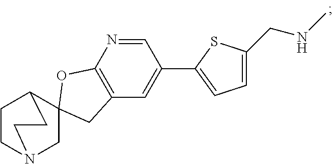 [SMILES: CNCc1ccc(-c2cnc3c(c2)CC2(CN4CCC2CC4)O3)s1]

E-24a: N-methyl-1-{5-[(2R)-3′H-spiro[4-azabicyclo[2.2.2]octane-2,2′-furo[2,3-b]pyridin]-5′-yl]-2-thienyl}methanamine;
E-24b: N-methyl-1-{5-[(2S)-3′H-spiro[4-azabicyclo[2.2.2]octane-2,2′-furo[2,3-b]pyridin-5′-yl]-2-thienyl}methanamine;
E-25a: 6-[(Anilinocarbonyl)amino]-N-[(3R)-1-azabicyclo[2.2.2]oct-3-yl]-1-benzothiophene-2-carboxamide;
E-25b: N-[(3R)-1-Azabicyclo[2.2.2]oct-3-yl]-6-({[(4-chlorophenyl)amino]carbonyl}amino)-1-benzothiophene-2-carboxamide;
E-25c: N-[(3R)-1-Azabicyclo[2.2.2]oct-3-yl]-6-({[(2-methoxyphenyl)amino]carbonyl}-amino)-1-benzothiophene-2-carboxamide;
E-25d: N-[(3R)-1-Azabicyclo[2.2.2]oct-3-yl]-6-({[(4-methoxyphenyl)amino]carbonyl}amino)-1-benzothiophene-2-carboxamide;
E-25e: N-[(3R)-1-Azabicyclo[2.2.2]oct-3-yl]-6-({[(2-phenylethyl)amino]carbonyl}amino)-1-benzothiophene-2-carboxamide;
E-25f: N-[(3R)-1-Azabicyclo[2.2.2]oct-3-yl]-6-({[(3-cyanophenyl)amino]carbonyl}amino)-1-benziophene-2-carboxamide;
E-25g: N-[(3R)-1-Azabicyclo[2.2.2]oct-3-yl]-6-({[(3-bromophenyl)amino]carbonyl}amino)-1-benzothiophene-2-carboxamide;
E-25h: N-[(3R)-1-Azabicyclo[2.2.2]oct-3-yl]-6-({[(2-elhoxyphenyl)amino]carbonyl)amino)-1-benzothiophene-2-carboxamide;
E-25i: N-[(3R)-1-Azbicyclo[2.2.2]oct-3-yl]-6-({[(4-(dimethylamino)phenyl)amino]-carbonyl)amino)-1-benzothiophene-2-carboxamide;
E-25j: N-(3R)-1-Azabicyclo[2.2.2]oct-3-yl]-6-({[(2-nitrophenyl)amino]carbonyl}amino)-1-benzothiophene-2-carboxamide;
E-25k: N-[(3R)-1-Azabicyclo[2.2.2]oct-3-yl]-6-({[(2,6-difluorophenyl)amino]carbonyl}-amino)-1-benzothiophene-2-carboxamide;
E-25l: N-[(3R)-1-Azabicyclo[2.2.2]oct-3-yl]-6-({[(2,4-dichlorophenyl)amino]carbonyl}-amino)-1-benzothiophene-2-carboxamide;
E-25m: N-[(3R)-1-Azabicyclo[2.2.2]oct-3-yl]-6-[({[3-(trifluoromethyl)phenyl]amino-carbonyl)amino]-1-benzothiophene-2-carboxamide;
E-25n: N-[(3R)-1-Azabicyclo[2.2.2]oct-3-yl]-6-({[(3,4,5-trimethoxyphenyl)amino]-carbonyl}amino)-1-benzothiophene-2-carboxamide;
E-25o: N-[(3R)-1-Azabicyclo[2.2.2]oct-3-yl]-8-[({[4-methoxy-3-(trifluoromethyl)phenyl]-amino}carbonyl)amino]-1-benzothiophene-2-carboxamide;
E-25p: N-{(3R)-1-Azabicyclo[2.2.2]oct-3-yl]-6-[({[3-methoxyphenyl]amino}carbonyl)-amino]-1-benzothiophene-2-carboxamide;
E-25q: N-[(3R)-1-Azabicyclo[2.2.2]oct-3-yl]-6-[({[3-trifluoromethoxyphenyl]amino}-carbonyl)-amino]-1-benzothiophene-2-carboxamide;
E-25r: N-[(3R)-1-Azabicyclo[2.2.2]oct-3-yl]-6-{[(tert-butylamino)carbonyl]amino}-1-benzothiophene-2-carboxamide;
E-25s: N-[(3R)-1-Azabicyclo[2.2.2]oct-3-yl]-6-{[(cyclohexylamino)carbonyl]amino}-1-benzothiophene-2-carboxamide;
E-25t: N-((3R)-1-Azabicyclo[2.2.2oct-3-yl-6-({[(1S)-1-phenylethyl]amino}carbonyl-amino]-1-benzothiophene-2-carboxamide;
E-25u: 7-[(Anilinocarbonyl)amino]-N-((3R)-1-azabicyclo[2.2.2oct-3-yl]-1-benzothiophene-2-carboxamide;
E-25v: N-[(3R)-1-Azabicyclo[2.2.2]oct-3-yl]-6-({[(4-methoxyphenyl)amino]carbonyl}-amino)-1-benzofuran-2-carboxamide;
E-26a: N-[4-(2-Thienyl)phenyl]-1-azabicyclo[2.2.2]octane-3-carboxamide;
E-26b: N-[4′-(Hydroxymethyl)-1,1′-biphenyl-4-yl]-1-azabicyclo[2.2.2]octane-3-carboxamide;
E-26c: N-(4-Fluoro-1,1′-biphenyl-4-yl)-1-azabicyclo[2.2.2]octane-3-carboxamide;
E-26d: N-(4′-Methylsulfanyl-1,1′-biphenyl-4-yl)-1-azabicyclo[2.2.2]octane-3-carboxamide;
E-26e: 2-(1-Azabicyclo[2.2.2]oct-3-yl)-N-(4′-fluoro-1,1′-biphenyl-4-yl)acetamide;
E-26f: 2-(1-Azabicyclo[2.2.2]oct-3-yl)-N-(4′-methoxy-1,1′-biphenyl-4-yl)acetamide;
E-26g: 2-(1-Azabicyclo[2.2.2]oct-3-yl)-N-(4′-fluoro-1,1′-biphenyl-3-yl)acetamide;
E-26h: 2-(1-Azabicyclo[2.2.2]oct-3-yl)-N-(3′-nitro-1,1′-biphenyl-4-yl)acetamide;
E-26i: 2-(1-Azabicyclo[2.2.2]oct-3-yl)-N-[4′-(hydroxymethyl)-1,1′-biphenyl-3-yl]acetamide;
E-26j: 2-(1-Azabicyclo[2.2.2]oct-3-yl)-N-[4′-(bromomethyl)-1,1-biphenyl-4-yl]acetamide;
E-26k: 2-(1-Azabicyclo[2.2.2]oct-3-yl)-N-[2′-(hydroxymethyl)-1,1′-biphenyl-3-yl]acetamide;
E-26i: N-[3′(Acetylamino)-1,1′-biphenyl-4-yl]-2-(1-azabicyclo[2.2.2]oct-3-yl)acetamide;
E-26m: (3R)—N-[2′-(Hydroxymethyl)-1,1′-biphenyl-4-yl]-1-azabicyclo[2.2.2]octane-3-carboxamide;
E-26n: (3R)—N-[4′-(Hydroxymethyl)-1,1-biphenyl-4-yl]-1-azabicyclo[2.2.2]octane-3-carboxamide;
E-26o: (3S)—N-[4′(Hydroxymethyl)-1,1′-biphenyl-4-yl]-1-azabicyclo[2.2.2]octane-3-carboxamide;
E-26p: (3R)—N-[4′-(4-Morpholinyl)-1,1′-biphenyl-4-yl]-1-azabicyclo[2.2.2]octane-3-carboxamide;
E-26q: (3R)—N-[4′-(Hydroxymethyl)-3′-(methoxy)-1,1′-biphenyl-4-yl]-1-azabicyclo[2.2.2]-octane-3-carboxamide;
E-26r: Methyl 4′-{[(3S)-1-azabicyclo[2.2.2]oct-3-ylcarbonyl]amino}-1,1′-biphenyl-4-carboxylate;
E-26s: 4′-{[(3S)-1-Azabicyclo[2.2.2]oct-3-ylcarbonyl]amino}-1,1′-biphenyl-4-carboxylic Acid;
E-26t: (3R)—N-[4′-(Hydroxy-1-methylethyl)-1,1′-biphenyl-4-yl]-1-azabicyclo[2.2.2]-octane-3-carboxamide;
E-26u: (3R)—N-[4′-(Aminocarbonyl)-1,1′-biphenyl-4-yl]-1-azabicyclo[2.2.2]octane-3-carboxamide;
E-26v: (3R)—N-[4′-(Hydroxymethyl)-3-fluoro-1,1′-biphenyl-4-yl]-1-azabicyclo[2.2.2]octane-3-carboxamide;
E-26w: (4′-{[(3R)-1-Azabicyclo[2.2.2]oct-3-ylcarbonyl]amino}-1,1′-biphenyl-4-yl)methyl Methylcarbamate;
E-26x: (4′-([(3R)-1-Azabicyclo[2.2.2]oct-3-ylcarbonyl]amino-1,1′-biphenyl-4-yl)methyl Isopropylcarbamate;
E-26y: (4′-{[(3R)-1-Azabicyclo[2.2.2]oct-3-ylcarbonyl]amino}-1,1′-biphenyl-4-yl)methyl Ethylcarbamate;
E-26z: the free base form of a compound being selected from Examples No 26, 27, 28, 29, 30, 31, 32, 33, 34 and 35 of WO2003/078431;
E-27a: 2-(1-Azabicyclo[2.2.2]oct-3-yl)-N-(7-bromo-1-benzothien-2-yl)acetamide;
E-27b: 2-(1-Azabicyclo[2.2.2]oct-3-yl)-N-(6-bromo-1-benzothien-2-yl)acetamide;
E-27c: 2-(1-Azabicyclo[2.2.2]oct-3-yl)-N-(7-quinolinyl)acetamide;
E-27d: 2-(1-Azabicyclo[2.2.2]oct-3-yl)-N-(2-naphthyl)acetamide;
E-27e: 2-(1-Azabicyclo[2.2.2]oct-3-yl)-N-(8-nitro-2-naphthyl)acetamide;
E-28a: N-(1-Azabicyclo[2.2.2]oct-3-yl)-6-quinolinecarboxamide;
E-28b: N-(1-Azabicyclo[2.2.2]oct-3-yl)-2-phenazinecarboxamide;
E-28c: N-(1-Azabicyclo[2.2.2]oct-3-yl)-7-quinolinecarboxamide;
E-28d: N-[(3R)-1-Azabicyclo[2.2.2]oct-3-yl]-6-quinolinecarboxamide;
E-28e: N-(1-Azabicyclo[2.2.2]oct-3-yl)-2-ethyl-7-quinolinecarboxamide;
E-28f: N-(1-Azabicyclo[2.2.2]oct-3-yl)-2-ethyl-6-quinolinecarboxamide;
E-28g: N-(1-Azabicyclo[2.2.2]oct-3-yl)-2-methyl-7-quinolinecarboxamide;
E-28h: N-(1-Azabicyclo[2.2.2]oct-3-yl)-2-methyl-6-quinoliecarboxamide;
E-28i: N-(1-Azabicyclo[2.2.2]oct-3-yl)-4-methyl-6-quinolinecarboxamide;
E-28j: N-(1-Azabicyclo[2.2.2]oct-3-yl)-2-propyl-6-quinolinecarboxamide;
E-28k: N-(1-Azabicyclo[2.2.2]oct-3-yl)-2-ethyl-4-methyl-6-quinolinecarboxamide;
E-281: N-(1-Azabicyclo[2.2.2]oct-3-yl)-2-propyl-7-quinolinecarboxamide;
E-28m: N-(1-Azabicyclo[2.2.2]oct-3-yl)-2-ethyl-4-methyl-7-quinolinecarboxamide;
E-28n: N-(1-Azabicyclo[2.2.2]oct-3-yl)-4-(tetrahydro-2H-pyran-2-yl)-6-quinoline-carboxamide;
E-28o: N-(1-Azabicyclo[2.2.2]oct-3-yl)-4-(tetrahydro-2H-pyran-2-yl)-7-quinoline-carboxamide;
E-28p: N-(1-Azabicyclo[2.2.2]oct-3-yl)-2-phenyl-6-quinolinecarboxamide;
E-28q: N-(1-Azabicyclo[2.2.2]oct-3-yl)-2-phenyl-7-quinolinecarboxamide;
E-29: (R)-7-chloro-N-(quinuclidin-3-yl)benzo[b]thiophene-2-carboxamide;
E-30a: 5-{5-[(endo)-8-azabicyclo[3.2.1]octan-3-yloxy]pyridin-2-yl}-H-indole;
E-30b: 5-{5-[(exo)-8-azabicyclo[3.2.1]octan-3-yloxy]pyridin-2-yl}-1H-indole;
E-30c: 5-{5-[(endo)-8-methyl-8-aza-bicyclo[3.2.1]oct-3-yloxy]pyridin-2-yl}-1H-indole;
E-30d: 5-{5-[(exo)-8-methyl-8-aza-bicyclo[3.2.1]oct-3-yloxy]pyridin-2-yl}-1H-indole; D-30e: 4-{5-[(exo)-8-methyl-8-aza-bicyclo[3.2.1]oct-3-yloxy]pyridin-2-yl}-H-indole; and
E-30f: 5-{6-[(exo)-8-methyl-8-aza-bicyclo[3.2.1]oct-3-yloxy]pyridin-3-yl}-1H-indole;
   wherein each of said compound is in free base form or in acid addition salt form.

In one embodiment, the α7-nAChR agonist is a compound selected from the group consisting of compound A-1, A-2 and A-3; wherein each of said compound is in free base form or in acid addition salt form.

In one embodiment, the α7-nAChR agonist is a compound selected from the group consisting of compound B-1, B-2, B-3, B-4, B-5, B-6, B-7, B-8, B-9, B-10, B-11, B-12, B-13, B-14, B-15, B-16, B-17, B-18, B-19, B-20 and B-21; wherein each of said compound is in free base form or in acid addition salt form.

In one embodiment, the α7-nAChR agonist is a compound selected from the group consisting of compound C-1, C-2, C-3, C-4, C-5, C-6, C-7, C-8, C-9, C-10, C-11 and C-12; wherein each of said compound is in free base form or in acid addition salt form.

In one embodiment, the α7-nAChR agonist is a compound selected from the group consisting of compound D-1, D-2, D-3 and D-4; wherein each of said compound is in free base form or in acid addition salt form.

In one embodiment, the α7-nAChR agonist is a compound selected from Group P2; Group P2 is the group consisting of compounds A-1, A-2, A-3, B-1, B-2, B-3, B-4, B-5, B-6, B-7, B-8, B-9, B-10, B-11, B-12, B-13, B-14, B-15, B-16, B-17, B-18, B-19, B-20, B-21, C-1, C-2, C-3, C-4, C-5, C-6, C-7, C-8, C-9, C-10, C-11, C-12, E-1, E-1a, E-1b, E-1c, E-1d, E-2, E-3, E-3a, E-4, E-4-a, E-8, E-8a, E-8b, E-9, E-9a, E-9b, E-10, E-10a, E-11, E-11a, E-11b, E-11c, E-11d, E-11e, E-12, E-19, E-22, E-24, E-24a, E-24b, E-25a, E-25b, E-25c, E-25d, E-25e, E-25f, E-25g, E-25h, E-25i, E-25j, E-25k, E-251, E-25m, E-25n, E-25o, E-25p, E-25q, E-25r, E-25s, E-25t, E-25u, E-25v, E-28a, E-28b, E-28c, E-28d, E-28e, E-28f, E-28g, E-28h, E-28i, E-28j, E-28k, E-281, E-28m, E-28n, E-28o, E-28p, E-28q, E-29, E-30a, E-30b, E-30c, E-30d, E-30e and E-30f; wherein each of said compound is in free base form or in acid addition salt form.

In one embodiment, the α7-nAChR agonist is a compound selected from Group P3; Group P3 is the group consisting of compounds A-1, A-2, A-3, B-1, B-2, B-3, B-4, B-5, B-6, B-7, B-8, B-9, B-10, B-11, B-12, B-13, B-14, B-15, B-16, B-17, B-18, B-19, B-20, B-21, C-1, C-2, C-3, C-4, C-5, C-6, C-7, C-8, C-9, C-10, C-11, C-12, E-1, E-1a, E-1b, E-1c, E-1d, E-2, E-3, E-3a, E-4, E-4-a, E-8, E-8a, E-8b, E-9, E-9a, E-9b, E-10, E-10a, E-11, E-11a, E-12, E-19, E-22, E-24, E-24a, E-24b, E-29, E-30a, E-30b, E-30c, E-30d, E-30e and E-30f; wherein each of said compound is in free base form or in acid addition salt form.

The compounds of formula (I) (e.g. compounds A-1 to A-3, B-1 to B-21 and C-1 to C-12) or compounds of formula (II) (e.g. compounds D-1 to D-4) and their manufacture are known from WO2001/85727, WO2004/022556, WO2005/118535, WO2005/123732, WO2006/005608, WO2007/045478, WO2007/068476 and WO2007/068475, or can be prepared analogously to said references.

Compounds E-1 and E-1a can be prepared according to WO2008/058096.

Compounds E-2, E-3, E-3a, E-4, E-4a and E-5 (A-582941) can be prepared according to WO2005/028477.

Compounds E-6, E-6a, E-7 and E7a can be prepared according to WO2006/065233 and/or WO2007/018738.

Compounds E-8, E-8a, E-8b, E-9, E-9a and E-9b can be prepared according to WO2004/029050 and/or WO2010/043515.

Compounds E-10 and E-10a can be prepared according to WO2004/076449 and/or WO2009/018505;

Compounds E-11, E-1a to E-11e can be prepared according to WO2004/076449 and/or WO2010/085724 and/or WO2010/056622;

Compounds E-12 (CP-810123) and Compound E-19 (varenicline) are described in O'Donnell et al, J Med Chem, 2010, 53, 1222-1237.

Compounds E-13 (PNU-282987), E-14 (PHA543613), E-21 (SSR-180771) and E-23 (ABBF) are described in Horenstein et al, Mol Pharmacol, 2008, 74, 1496-1511.

Compounds E-15 (PHA568487), E-16 (WAY-317538), E-17 (WAY-264620), E-20 (AZD-0328) and E-22 (GTS-21) are described in Haydar et al, Current Topics in Medicinal Chemistry, 2010, 10, 144-152.

Compound E-18 (WYE-103914) is described in Ghiron et al, J Med Chem, 2010, 53, 4379-4389.

Compound E-24, E-24a and E-24b are described in WO2007/133155 and/or WO2009/066107.

Compounds E-25a to E-25v are described in WO2004/013136.

Compounds E-26a to E-26z are described in WO2003/078431.

Compounds E-27a to E-27e are described in WO2003/078430.

Compounds E-28a to E-28q are described in WO2003/043991.

Compound E-29 is described in WO2003/055878.

Compounds D-30a to D-30f are described in WO2007/137030.

A further aspect of the invention concerns the use of a α7-nAChR agonist for the treatment (whether therapeutic or prophylactic), prevention or delay of progression of an Instant Movement Disorder; wherein said α7-nAChR agonist is a compound of formula (I).

A further aspect of the invention concerns the use of a α7-nAChR agonist for the treatment (whether therapeutic or prophylactic), prevention or delay of progression of an Instant Movement Disorder; wherein said α7-nAChR agonist is a compound selected from the Group P1.

A further aspect of the invention concerns the use of a α7-nAChR agonist for the treatment (whether therapeutic or prophylactic), prevention or delay of progression of an Instant Movement Disorder; wherein said α7-nAChR agonist is a compound selected from the Group P2.

A further aspect of the invention concerns the use of a α7-nAChR agonist for the treatment (whether therapeutic or prophylactic), prevention or delay of progression of an Instant Movement Disorder; wherein said α7-nAChR agonist is a compound selected from the Group P3.

A further aspect of the invention relates to a method for the treatment, prevention or delay of progression of an Instant Movement Disorder in a subject in need of such treatment, which comprises administering to said subject a therapeutically effective amount of a α7-nAChR agonist; wherein said α7-nAChR agonist is a compound of formula (I).

A further aspect of the invention relates to a method for the treatment, prevention or delay of progression of an Instant Movement Disorder in a subject in need of such treatment, which comprises administering to said subject a therapeutically effective amount of a α7-nAChR agonist; wherein said α7-nAChR agonist is a compound selected from the Group P1.

A further aspect of the invention relates to a method for the treatment, prevention or delay of progression of an Instant Movement Disorder in a subject in need of such treatment, which comprises administering to said subject a therapeutically effective amount of a α7-nAChR agonist; wherein said α7-nAChR agonist is a compound selected from the Group P2.

A further aspect of the invention relates to a method for the treatment, prevention or delay of progression of an Instant Movement Disorder in a subject in need of such treatment, which comprises administering to said subject a therapeutically effective amount of a α7-nAChR agonist; wherein said α7-nAChR agonist is a compound selected from the Group P3.

A further aspect of the invention relates to a method for the treatment, prevention or delay of progression of an Instant Movement Disorder in a subject in need of such treatment, which comprises (i) diagnosing said Movement Disorder in said subject and (ii) administering to said subject a therapeutically effective amount of a α7-nAChR; wherein said α7-nAChR agonist is a compound of formula (I).

A further aspect of the invention relates to a method for the treatment, prevention or delay of progression of an Instant Movement Disorder in a subject in need of such treatment, which comprises (i) diagnosing said Movement Disorder in said subject and (ii) administering to said subject a therapeutically effective amount of a α7-nAChR; wherein said α7-nAChR agonist is a compound selected from the Group P1.

A further aspect of the invention relates to a method for the treatment, prevention or delay of progression of an Instant Movement Disorder in a subject in need of such treatment, which comprises (i) diagnosing said Movement Disorder in said subject and (ii) administering to said subject a therapeutically effective amount of a α7-nAChR; wherein said α7-nAChR agonist is a compound selected from the Group P2.

A further aspect of the invention relates to a method for the treatment, prevention or delay of progression of an Instant Movement Disorder in a subject in need of such treatment, which comprises (i) diagnosing said Movement Disorder in said subject and (ii) administering to said subject a therapeutically effective amount of a α7-nAChR; wherein said α7-nAChR agonist is a compound selected from the Group P3.

A further aspect of the invention relates to a pharmaceutical composition comprising a α7-nAChR agonist for the treatment, prevention or delay of progression of an Instant Movement Disorder; wherein said α7-nAChR agonist is a compound of formula (I).

A further aspect of the invention relates to a pharmaceutical composition comprising a α7-nAChR agonist for the treatment, prevention or delay of an Instant Movement Disorder; wherein said α7-nAChR agonist is a compound selected from the Group P1.

A further aspect of the invention relates to a pharmaceutical composition comprising a α7-nAChR agonist for the treatment, prevention or delay of progression of an Instant Movement Disorder; wherein said α7-nAChR agonist is a compound selected from the Group P2.

A further aspect of the invention relates to a pharmaceutical composition comprising a α7-nAChR agonist for the treatment, prevention or delay of progression of an Instant Movement Disorder; wherein said α7-nAChR agonist is a compound selected from the Group P3.

A further aspect of the invention relates to the use of a α7-nAChR agonist for the manufacture of a medicament for the treatment, prevention or delay of progression of an Instant Movement Disorder; wherein said α7-nAChR agonist is a compound of formula (I).

A further aspect of the invention relates to the use of a α7-nAChR agonist for the manufacture of a medicament for the treatment, prevention or delay of progression of an Instant Movement Disorder; wherein said α7-nAChR agonist is a compound selected from the Group P1.

A further aspect of the invention relates to the use of a α7-nAChR agonist for the manufacture of a medicament for the treatment, prevention or delay of progression of an Instant Movement Disorder; wherein said α7-nAChR agonist is a compound selected from the Group P2.

A further aspect of the invention relates to the use of a α7-nAChR agonist for the manufacture of a medicament for the treatment, prevention or delay of progression of an Instant Movement Disorder; wherein said α7-nAChR agonist is a compound selected from the Group P3.

### Nicotinic Acetylcholine Receptor Alpha 7 Positive Allosteric Modulator:

As used herein a “α7-nAChR positive allosteric modulator” is a compound that binds to a receptor comprising a α7-nAChR subunit in vivo and in vitro and is potentiating the activation of the receptor when its physiological ligand (i.e. acetylcholine) is binding. Potentiation can be measured by the method disclosed in WO2001/85727, i.e. a functional affinity assay at the homomeric alpha 7 nicotinic acetylcholine receptor (α7 nAChR) carried out with a rat pituitary cell line stably expressing the α7 nAChR. As read out, the calcium influx upon stimulation of the receptor compared to acetylcholine-binding alone is used. “α7-nAChR positive allosteric modulators” according to the invention typically induce calcium influx of at least 200% of the maximal influx evoked by acetylcholine with an EC o value of at least 5000 nM; preferred agonists induce calcium influx of at least 300% of the maximal influx evoked by acetylcholine with an EC₅₀ value of at least 1000 nM; more preferred agonists induce calcium influx of at least 400% of the maximal influx evoked by epibatidine with an EC₅₀ value of at least 500 nM.

In particular, preferred α7-nAChR positive allosteric modulators should be well absorbed from the gastrointestinal tract, should be sufficiently metabolically stable and possess favorable pharmacokinetic properties.

Further preferred α7-nAChR positive allosteric modulators bind in-vivo potently to α7-nAChRs whilst showing little affinity for other receptors, especially for other nAChRs, e.g. α 4β2 nAChR, for muscarinic acetylcholine receptors, e.g. M1, and/or the 5-HT₃ receptor.

Further preferred α7-nAChR positive allosteric modulators cross the blood brain barrier effectively.

Preferred α7-nAChR positive allosteric modulators should be non-toxic and demonstrate few side-effects.

Furthermore, a preferred α7-nAChR positive allosteric modulator will be able to exist in a physical form that is stable, non-hygroscopic and easily formulated.

In one embodiment, the α7-nAChR positive allosteric modulator is selective for a receptor comprising a α7-nAChR subunit, since such a positive allosteric modulator would be expected to cause fewer side effects than a non-selective positive allosteric modulator to a treated subject. A positive allosteric modulator being selective for a receptor comprising a α7-nAChR subunit has a functional affinity to such a receptor to a much higher degree, e.g. at least 10-fold affinity difference in EC₅₀ value, preferably at least 20-fold, more preferably at least 50-fold, compared to any other nicotinic acetylcholine receptor. To assess the affinity of the α7-nAChR positive allosteric modulator of the invention on other nicotinic acetylcholine receptors, the method disclosed in WO2001/85727 can be used, i.e. to assess the affinity on human neuronal α4β2 nAChR, a similar functional assay is carried out using a human embryonic kidney cell line stable expressing the human α4β2 subtype and to assess the activity of the compounds of the invention on the “ganglionic subtype” and the “muscle type” of nicotinic receptor, similar functional assays are carried out with a human embryonic kidney cell line stably expressing the human “ganglionic subtype” or a cell line endogenously expressing the human “muscle type” of nicotinic receptors.

In the last 12 years much effort has been focused on developing selective α7 nAChR positive allosteric modulators leading to the discovery of many different chemotypes displaying said selective activity. These efforts are summarized the review from Haydar et al (Current Topics in Medicinal Chemistry, 2010, 10, 144-152), which describes 11 compounds acting as α7 nAChR positive allosteric modulators belonging to seven different chemical families; i.e. XY-4083; PNU-120596, PHA-758454 and NS-1738; PHA-709829; SB-206553; LY-2087101, LY-1078733 and LY-2087133; compound 26; and A-867744 (compound designations taken from Haydar et al). All said 11 compounds described in Haydar et al are incorporated herein by reference. In fact, at least one drug candidate having an α7 nAChR positive allosteric modulator mode of action obtained permission from the U.S. Food and Drug Administration to conduct clinical testing (i.e. XY-4083).

In one embodiment, the α7-nAChR positive allosteric modulator is a low molecular weight compound.

In one embodiment, the α7-nAChR positive allosteric modulator has a maximum molecular weight of 1500 daltons.

In one embodiment, the α7-nAChR positive allosteric modulator has a maximum molecular weight of 1000 daltons.

In one embodiment, the α7-nAChR positive allosteric modulator has a maximum molecular weight of 800 daltons.

In one embodiment, the α7-nAChR positive allosteric modulator has a maximum molecular weight of 500 daltons.

In one embodiment, the α7-nAChR positive allosteric modulator is a compound selected from the Group P4; Group P4 is the group consisting of compounds
F-1: (Z)—N-(4-Chloro-phenyl)-3-(4-chloro-phenylamino)-2-(3-methyl-isoxazol-5-yl)-acrylamide (XY-4083);
F-2: 1-(5-Chloro-2,4-dimethoxy-phenyl)-3-(5-methyl-isoxazol-3-yl)-urea (PNU-120596);
F-3: 1-(5-Fluoro-2,4-dimethoxy-phenyl)-3-(5-trifluoromethyl-isoxazol-3-yl)-urea (PHA-758454);
F-4: 1-(5-Chloro-2-hydroxy-phenyl)-3-(2-chloro-5-trifluoromethyl-phenyl)-urea (NS-1738);
F-5: 4-(4-Chloro-phenyl)-2-(4-methoxy-phenyl)-5-methyl-2H-pyrazol-3-ylamine (PHA-709829);
F-6: 5-Methyl-3,5-dihydro-2H-pyrrolo[2,3-f]indole-1-carboxylic acid pyridin-3-ylamide (SB-206553);
F-7: [2-(4-Fluoro-phenylamino)-4-methyl-thiazol-5-yl]-thiophen-3-yl-methanone (LY-2087101);
F-8: [2-(4-Fluoro-phenylamino)-4-methyl-thiazol-5-yl]-p-tolyl-methanone (LY-1078733);
F-9: Benzo[1,3]dioxol-5-yl-[2-(4-fluoro-phenylamino)-4-methyl-thiazol-5-yl]-methanone (LY-2087133);
F-10: 4-Naphthalen-1-yl-3a,4,5,9b-tetrahydro-3H-cyclopenta[c]quinoline-8-sulfonic acid amide; and
F-11: 4-[5-(4-Chloro-phenyl)-2-methyl-3-propionyl-pyrrol-1-yl]-benzenesulfonamide (A-867744);
   wherein said compound is in free base form or in acid addition salt form.

A further aspect of the invention concerns the use of a α7-nAChR positive allosteric modulator for the treatment (whether therapeutic or prophylactic), prevention or delay of progression of an Instant Movement Disorder; wherein said α7-nAChR positive allosteric modulator is a compound selected from the Group P4.

A further aspect of the invention relates to a method for the treatment, prevention or delay of progression of an Instant Movement Disorder in a subject in need of such treatment, which comprises administering to said subject a therapeutically effective amount of a α7-nAChR positive allosteric modulator; wherein said α7-nAChR positive allosteric modulator is a compound selected from the Group P4.

A further aspect of the invention relates to a method for the treatment, prevention or delay of progression of an Instant Movement Disorder in a subject in need of such treatment, which comprises (i) diagnosing said Movement Disorder in said subject and (ii) administering to said subject a therapeutically effective amount of a α7-nAChR positive allosteric modulator; wherein said α7-nAChR positive allosteric modulator is a compound selected from the Group P4.

A further aspect of the invention relates to a pharmaceutical composition comprising a α7-nAChR positive allosteric modulator for the treatment, prevention or delay of progression of an Instant Movement Disorder; wherein said α7-nAChR positive allosteric modulator is a compound selected from the Group P4.

A further aspect of the invention relates to the use of a α7-nAChR positive allosteric modulator for the manufacture of a medicament for the treatment, prevention or delay of progression of an Instant Movement Disorder; wherein said α7-nAChR positive allosteric modulator is a compound selected from the Group P4.

Salt Forms of α7-nAChR Agonists or α7-nAChR Positive Allosteric Modulators:

The acid addition salt of α7-nAChR agonists or α7-nAChR positive allosteric modulators are preferably pharmaceutically acceptable salts. Such salts are known in the field (e.g. S. M. Berge, et al, “Pharmaceutical Salts”, J. Pharm. Sd., 1977, 66:1-19; and “Handbook of Pharmaceutical Salts, Properties, Selection, and Use”, Stahl, R H., Wermuth, C. G., Eds.; Wiley-VCH and VHCA: Zurich, 2002). A “pharmaceutically acceptable salt” is intended to mean a salt of a free base of a α7-nAChR agonist or α7-nAChR positive allosteric modulator that is not toxic, biologically intolerable, or otherwise biologically undesirable. Preferred pharmaceutically acceptable salts are those that are pharmacologically effective and suitable for contact with the tissues of patients without undue toxicity, irritation, or allergic response.

### Pharmaceutical Compositions:

For use according to the invention, the α7-nAChR agonist or α7-nAChR positive allosteric modulator may be administered as single active agent or in combination with other active agents, in any usual manner, e.g. orally, for example in the form of tablets or capsules, parenterally, for example in the form of injection solutions or suspensions, or transdermally, for example in the form of a patch.

In one embodiment, the manner of administration is oral administration, for example in the form of tablets or capsules.

In one embodiment, the manner of administration is transdermal administration, for example in the form of a patch.

Moreover, the present invention provides a pharmaceutical composition comprising a α7-nAChR agonist or α7-nAChR positive allosteric modulator in association with at least one pharmaceutical carrier or diluent for the treatment, prevention or delay of progression of the Instant Movement Disorder. Such compositions may be manufactured in conventional manner. Unit dosage forms may contain, for example, from about 2.5 to about 25 mg of one or more of the α7-nAChR agonist or α7-nAChR positive allosteric modulator.

The pharmaceutical compositions according to the invention are compositions for enteral, such as nasal, rectal or oral; parenteral, such as intramuscular or intravenous; or transdermal (e.g. by a patch) administration to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes. Such processes are exemplified in WO 2005/079802, WO 2003/047581, WO 2004/000316, WO 2005/044265, WO 2005/044266, WO 2005/044267, WO 2006/114262 and WO 2007/071358.

Compositions for transdermal administration are described in Remington's Pharmaceutical Sciences 16^{th} Edition Mack; Sucker, Fuchs and Spieser, Pharmazeutische Technologie, 1^{st} Edition, Springer.

The usefulness of the α7-nAChR agonists or α7-nAChR positive allosteric modulators in the treatment of the above-mentioned disorders can be confirmed in a range of standard tests including those indicated below.

### 1. In-Vitro Tests

1.1. Selectivity of Selected α7-nAChR Agonists Against α4β2-nAChR

Based on the activity/selectivity data shown below it is concluded that said compounds are selective agonists at the α7-nAChR.

| | α7-nAChR activity | | | | |
|---|---|---|---|---|---|
| | | Efficacy | | | |
| | Potency | compared to | α4β2-nAChR activity | | |
| | EC₅₀ | epibatidine | IC₅₀ | EC₅₀ | fold |
| Compound | (nM) | (100%) | (nM) | (nM) | selectivity |
| A-1 | 100 | 83 | 23442 | >100 000 | 234 |
| C-1 | 24 | 84 | 9333 | >100 000 | 388 |
| B-13 | 13 | 89 | 4217 | >100 000 | 324 |

### Assay:

To assess α7-nAChR activity, a functional assay was employed using GH3 cells that recombinantly expressed human α7-nAChR. 50000 cells per well were seeded 72 h prior to the experiment on black 96-well plates (Costar) and incubated at 37° C. in a humidified atmosphere (5% CO₂/95% air). On the day of the experiment, medium was removed by flicking the plates and replaced with 100 μl growth medium containing 2 mM Fluo-4, (Molecular Probes) in the presence of 2.5 mM probenecid (Sigma). The cells were incubated at 37° C. in a humidified atmosphere (5% CO2/95% air) for 1 h. Plates were flicked to remove excess of Fluo-4, washed twice with Hepes-buffered salt solution (in mM: NaCl 130, KCl 5.4, CaCl2 2, MgSO4 0.8, NaH2PO4 0.9, glucose 25, Hepes 20, pH 7.4; HBS) and refilled with 100 μl of HBS containing antagonist when appropriate. The incubation in the presence of the antagonist lasted 3-5 minutes. Plates were placed in the cell plate stage of a FLIPR device (fluorescent imaging plate reader, Molecular Devices, Sunnyvale, Calif., USA). After recording of the baseline (laser: excitation 488 nm at I W, CCD camera opening of 0.4 seconds) the agonists (50 μl) were added to the cell plate using the FLIPR 96-tip pipettor while simultaneously recording the fluorescence. Calcium kinetic data were normalized to the maximal fitted response induced by epibatidine, which is a full agonist at α7-nAChR. Four parameter Hill equations were fitted to the concentration-response. Values of Emax (maximal effect in % compared to the epibatidine response) and EC50 (concentration producing half the maximal effect in μM) were derived from this fit.

Assay described in: D Feuerbach et al, Neuropharmacology (2005), 48, 215-227.

To assess the activity of the compound of the invention on the human neuronal nAChR α4β2, a similar functional assay is carried out using a human epithelial cell line stably expressing the human α4β2 subtype (Michelmore et al., Naunyn-Schmiedeberg's Arch.

Pharmacol. (2002) 366, 235).

### 2. In-Vivo Preclinical Tests

### 2.1. Oral Bioavailability and Brain Penetration in Mice

Based on the pharmacokinetic data shown below it is concluded that the brain concentration of said compounds in mice is beyond (or at least equal) to the compound's EC₅₀ at the α7-nAChR for at least 4 hours following an acute oral dose of 30 μmol/kg.

### Compound A-1:

| | | Plasma | Brain | Ratio |
|---|---|---|---|---|
| | Time | (pmoles/ | (pmoles/ | Brain/ |
| Administration | (hour) | ml ± SD) | g ± SD) | plasma |
| 30 μmol/kg p.o. | 0.5 | 634.9 ± 261.3 | 706.3 ± 153.4 | 1.1 |
| 30 μmol/kg p.o. | 1 | 684.7 ± 339.6 | 573.7 ± 109.3 | 0.8 |
| 30 μmol/kg p.o. | 2 | 168.2 ± 91.3 | 191.9 ± 34.9 | 1.1 |
| 30 μmol/kg p.o. | 4 | 85.0 ± 54.3 | 101.6 ± 39.6 | 1.2 |
| 30 μmol/kg p.o. | 6 | 29.5 ± 13.8 | 40.5 ± 12.1 | 1.4 |
| 30 μmol/kg p.o. | 24 | 3.8 ± 0.6 | 9.1 ± 2.7 | 2.4 |

### Compound B-13:

| | | Plasma | Brain | Ratio |
|---|---|---|---|---|
| | Time | (pmoles/ | (pmoles/ | Brain/ |
| Administration | (hour) | ml ± SD) | g ± SD) | plasma |
| 30 μmol/kg p.o. | 0.25 | 2196 ± 397 | 1884 ± 291 | 0.86 |
| 30 μmol/kg p.o. | 0.5 | 2265 ± 419 | 2960 ± 706 | 1.31 |
| 30 μmol/kg p.o. | 1 | 1554 ± 523 | 2940 ± 335 | 1.89 |
| 30 μmol/kg p.o. | 2 | 1172 ± 252 | 1260 ± 172 | 1.07 |
| 30 μmol/kg p.o. | 4 | 429 ± 167 | 379 ± 134 | 0.88 |
| 30 μmol/kg p.o. | 8 | 80 ± 23 | 93 ± 30 | 1.17 |
| 30 μmol/kg p.o. | 24 | * | 13 ± 4 | |

### Compound C-1:

| | | Plasma | Brain | Ratio |
|---|---|---|---|---|
| | Time | (pmoles/ | (pmoles/ | Brain/ |
| Administration | (hour) | ml ± SD) | g ± SD) | plasma |
| 30 μmol/kg p.o. | 0.25 | 1601 ± 758 | 620 ± 221 | 0.39 |
| 30 μmol/kg p.o. | 0.5 | 3414 ± 956 | 1405 ± 539 | 0.41 |
| 30 μmol/kg p.o. | 1 | 1241 ± 583 | 1458 ± 189 | 1.17 |
| 30 μmol/kg p.o. | 2 | 875 ± 261 | 1478 ± 259 | 1.69 |
| 30 μmol/kg p.o. | 4 | 762 ± 159 | 842 ± 187 | 1.11 |
| 30 μmol/kg p.o. | 8 | 239 ± 27 | 362 ± 62 | 1.51 |
| 30 μmol/kg p.o. | 24 | * | * | |

### Assay:

Compounds were orally (30 μmol/kg) administered. Male mice (30-35g, OF1/ICstrain) were sacrificed at indicated time points after oral administration. Trunk-blood was collected in EDTA-containing tubes and the brain was removed and immediately frozen on dry ice.

To 100 μl plasma 10 μl internal standard (1.0 pmol of a compound with solubility and ionization properties similar to test compounds) was added and extracted three times with 500 μl dichloromethane. The combined extracts were then dried under a stream of nitrogen and re-dissolved in 100 μl acetonitrile/water (70% acetonitrile). Brains were weighed and homogenized in water (1:5 w/v). Two 100 μl aliquots of each homogenate+10 μl of internal standard (same standard as used for the plasma samples) were extracted three times with 500 μl dichloromethane and further processed as the plasma samples. Samples were separated on Beckmann high-performance liquid chromatography equipment system with an autosampler (Gilson 233XL). A 10 min linear gradient (10-70%) of acetonitrile containing 0.5. % (v/v) formic acid was used to elute the compounds from Nucleosil CC-125/2 C18 reversed phase (Machery&Nagel) column.

The limit of detection (LOD), defined as the lowest concentration of the extracted standard sample with a signal to noise ratio of ˜3.

### 2.2. Functional Read-Out in Mice (Social Recognition Test)

Based on the functional in-vivo data shown below it is concluded that oral dosing of said compounds at relevant concentrations lead to a specific effect associated with α7-nAChR (i.e. cognition enhancement in the Social Recognition Test in mouse).

| | Reduction in time | Dose |
|---|---|---|
| Compound | scrutinizing in % ± SEM at 24 h | in mg/kg |
| A-1 | 52 ± 4 | 3 |
| C-1 | 51 ± 3 | 0.3 |
| B-13 | 37 ± 7 | 0.3 |

### Assay:

Social interactions between two experimental animals are influenced by their degree of familiarity: the better they know each other, the less time they spend on mutual scrutiny at each meeting. In agreement with published data in rats (Mondadori et al., 1993) we have observed (i) that an adult mouse shows a shortened scrutiny of a young conspecific if the two mice are brought together again within a short time interval (e.g. 1 hour), (ii) that this curtailment is attributable to memory processes: it does not occur if the familiar young partner is replaced by a strange (unfamiliar) young mouse on the second occasion and (iii) that the adult mouse's recollection of the previously scrutinized juvenile partner fades with the elapsed time, i.e., after 24 h, scrutiny takes just about as long as at the first encounter. Memory enhancing agents (i.e. oxiracetam) facilitate learning to the extent that the previously met (familiar) partner is still remembered after 24 h, whereas in vehicle treated control animals the memory usually fades after less than 1 hour (Thor and Holloway, 1982) or after 2-3 hours.

Baseline-test: Pairs consisting of one adult and one young mouse were assigned at random to the experimental and control groups. In each pair only the adult mouse was orally treated 1 hour before the trial with either vehicle or the test compound. The duration of active contacts of the adult mouse with the young mouse was manually recorded over a period of 3 min, including the following behavioural, approach-related items: sniffing, nosing, grooming, licking, pawing and playing, anogenital exploration and orientation toward the young mouse; orientation, thereby, was defined as tip of nose of the adult mouse less than approximately 1 cm distant from the young mouse's body.

Re-test: Twenty-four hours after the baseline-test, the adults in each treatment group were confronted again with the previously encountered (familiar) partner, whereas the half of the adult animals were put together with the previously encountered (familiar) partner and the other half with another (unfamiliar) young mouse. Again the duration of active approach-behaviours was recorded during a 3-min period. Prior to re-test no oral injection was given. In the table the reduction in time scrutinizing the familiar partner at time 24 compared with the familiar partner at time 0 minutes is given (value of zero would signify no reduction).

### 2.3. Assessment of Efficacy in a Movement Disorder Model (Antidyskinetic Effect in Parkinsonian Primates)

Based on the in-vivo data in movement-deficit primates (parkinsonian primates) shown below it is deduced that:

i) compound A-1 significantly reduces the deficiencies associated with movement disorders (e.g. compound A-1 significantly reduces levodopa-induced dyskinesia); and
ii) compound A-1 significantly increases the duration of antiparkinsonian activity associated with administering a combination of a dopamine agonist and compound A-1 (e.g. compound A-1 significantly increases the duration of the antiparkinsonian activity seen for levodopa administration).

It is further noted that compound A-1 does not delay the onset of action of levodopa and does not lower the antiparkinsonian activity of levodopa.

### 2.3.1 Method

Female ovariectomized cynomolgus monkeys (Macaca fascicularis) are used in the assessment. The animals can be rendered parkinsonian by continuous infusion of 1-methyl-4-phenyl-1,2,3,6-tetrahydropyridine (MPTP) until they develop a stable parkinsonian syndrome. After recuperation, animals are treated daily with levodopa until clear and reproducible dyskinesias are developed.

### 2.3.2 Assessment

Monkeys are observed through a one-way screen window in their home cage. They are observed and scored repeatedly at baseline and after a standard s.c. dose of levodopa. Locomotor activity is assessed and followed with an electronic monitoring system. Antiparkinsonian responses are evaluated by measuring the locomotor activity and a Parkinson disability scale (see Hadj Tahar A et al, Clin Neuropharmacol 2000; 23:195-202; and Samadi P et al, Neuropharmacology 2003; 45:954-963). Dyskinesias are closely monitored and scored according to a dyskinesia rating scale (also described in Hadj Tahar A et al; and Samadi P et al) every 15 minutes until the end of the effect. The doses of levodopa are chosen to induce motor activation and reproducible dyskinesia but no excessive agitation.

### 2.3.3 Protocol

Monkeys are observed for at least two hours following an oral administration of vehicle. On a subsequent day, the dose of levodopa selected is tested once. The animals are observed (with measures of parkinsonian and dyskinetic scores) for the entire duration of the levodopa effect and are also monitored for locomotor activity. This provides vehicle control values as well as levodopa antiparkinsonian and dyskinesia response data for comparison with combinations of a α7-nAChR agonist/positive allosteric modulator and levodopa. The monkeys are then tested with a α7-nAChR agonist/positive allosteric modulator in combination with a fixed dose of levodopa. A suspension for oral administration of the α7-nAChR agonist/positive allosteric modulator is administered before levodopa. After each dose, the animals are observed (with measures of parkinsonian and dyskinetic scores) for the entire duration of effect and monitored for locomotor activity or any change in behavior (e.g. circling, excitement, lethargy and sleepiness).

Using this protocol, compound A-1 at a dose of 20 mg/kg was tested. Results based on five monkeys (levodopa/benserazide doses: 22.5/50 mg; 65/50 mg; 30/50 mg; 35/50 mg; and 25/50 mg) are shown in FIGS. 1-4. In said experiments, compound A-1 reduced the Mean Dyskinesia Score (total period) from 2.8 to 2.1; furthermore, compound A-1 extended the Duration of Levodopa-Response from 230 minutes to 265 minutes. Neither Elapsed Time after Levodopa Administration or extent of the antiparkinsonian activity of Levodopa measured with the antiparkinsonian score were changed significantly with the addition of compound A-1.

### 2.4. Other Movement Disorder Models

Further, the α7-nAChR agonists/positive allosteric modulators may be tested in the following in-vivo models for movement disorders.

Simple/complex tics: assessing the improvement of iminodipropionitrile induced head twitch response in rats by administration of α7-nAChR agonists/positive allosteric modulators of invention (Diamond et al, Adv Neurol, 35, 1982, 221-225).

Restless legs syndrome: iron deprivation (ID) plus bilateral 6-hydroxydopamine (6-OHDA) lesions in the A11 nuclei in C57BL/6 mice increases locomotor activities which may be lessened by application of α7-nAChR agonists/positive allosteric modulators of invention (Luo et al, Sleep Med, 1, 2011, 41-46).

There are several animal assays that model different features of Parkinsonism pathophysiology (e.g. Symptomatic Parkinsonism, e.g. Drug-induced Parkinsonism, Parkinsonism caused by Diffuse Lewy Body Disorder or Parkinsonism caused by Multiple System Atrophy, e.g. Parkinsonism caused by Striatonigral Degeneration).

Administration of toxins like 6-OHDA, MTPT, rotenone, paraquat and reserpine to rats or monkeys leads to locomotor deficits that may be rescued by α7-nAChR agonists/positive allosteric modulators of invention. LRRK2, Pitx3-aphakia, MitoPark, and VMAT2-deficient mice replicate some of the phenotypes seen in familial Parkinson Disease which may be improved after administration of α7-nAChR agonists/positive allosteric modulators of invention. Literature: Taylora et al, Behavioural Brain Research, 211, 2010, 1-10; Lane et al, Psychopharmacology, 199, 2008, 303-312.

Alpha-synuclein transgenic animal models recapitulate some of the symptoms that are seen in the pathophysiology of Parkinsonism caused by Diffuse Lewy Body Disorder (like alpha-synuclein aggregates) and may be used to assess effects of administration of α7-nAChR agonists/positive allosteric modulators of invention. Literature: Crews et al, PLoS One, 5(2), 2010, e9313.

### 3. Clinical Testing: Improvement Trials

Clinical testing of the α7-nAChR agonist/positive allosteric modulator may be conducted, for example, in one of the following study designs. The skilled physician may look at a number of aspects of patient behaviors and abilities. He will realize that such studies are considered as guidelines and the certain aspects of the studies may be modified and redefined depending on the circumstance and environment, for example.

### 3.1 Trial A: Normal Patient Population

A patient population, with a normal control is dosed once a day for a week or longer tested. The test is designed to allow for improvement, i.e. that there is a measurable parameter increase of the impaired function. Patients are tested at the beginning and at the end of the dosage period and the results are compared and analyzed.

### 3.2 Trial B: Deficit Population

A patient population with a deficit associated with an Instant Movement Disorder, e.g. Restless Legs Syndrome, is dosed once a day for a week or longer and tested. The test is designed to allow for improvement, i.e. that there is a measurable parameter increase of the impaired function. The patients are tested at the beginning and at the end of the dosage period and the results are compared and analyzed.

### 3.3 Considerations for Designing a Trial

When designing a trial, the skilled person will appreciate the need to protect both against floor and ceiling effects. In other words, the study designing should allow cognition to the measurably raised or lowered.
Conditions that artificially impair a function, e.g. cognition, are one way to test enhancement of that function. Such conditions are, for example, sleep deprivation and pharmacological challenges.
Placebo control is required for all trials.
In assessing the data, evaluation of the likelihood of learning and practice effects from repeat assessments must be made. The likelihood of such effects contaminating the data to produce false positives should be taken in to account when designing the test, e.g. the tests should not be identical (e.g. commit the same list of words to memory) but designed to study the same mechanism. Other countermeasures may include single testing at the end of a trial only.

### DESCRIPTION OF FIGURES

FIG. 1: Elapsed time after L-dopa administration for behavioural response in parkinsonian primates

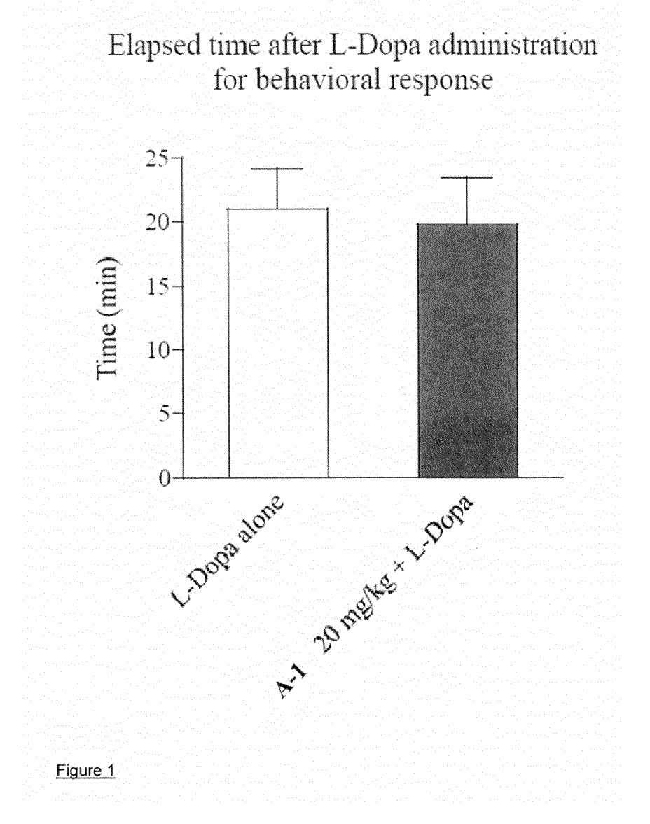

FIG. 2: Mean Parkinsonian Score (total period) after L-dopa administration in parkinsonian primates

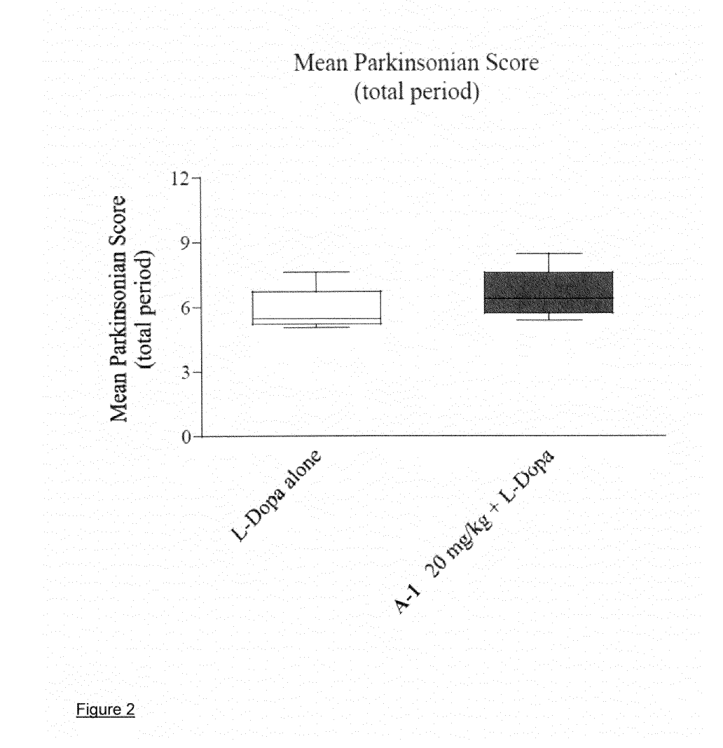

FIG. 3: Mean Dyskinesia Score (total period) after L-dopa administration in parkinsonian primates

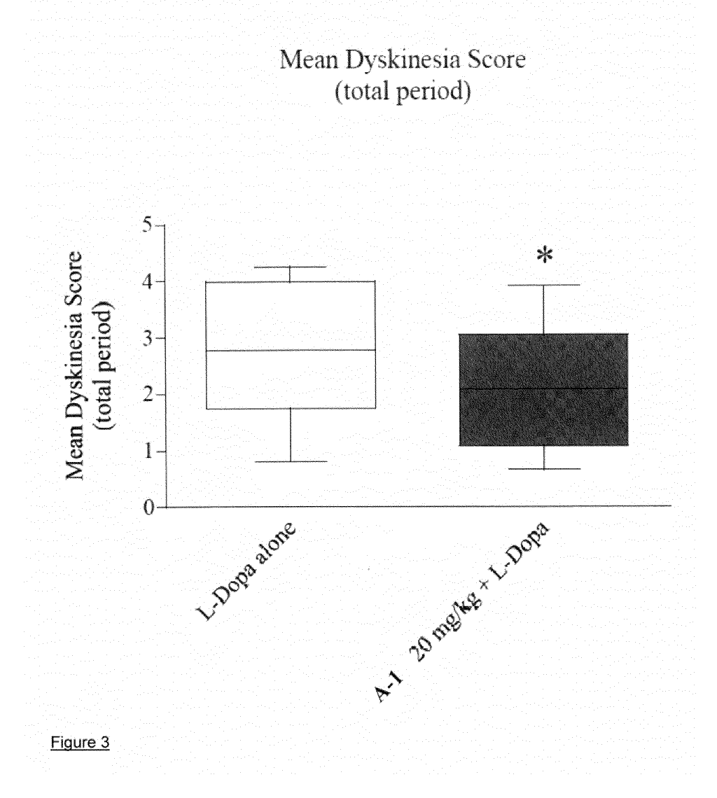

FIG. 4: Duration of L-dopa response after L-dopa administration in parkinsonian primates

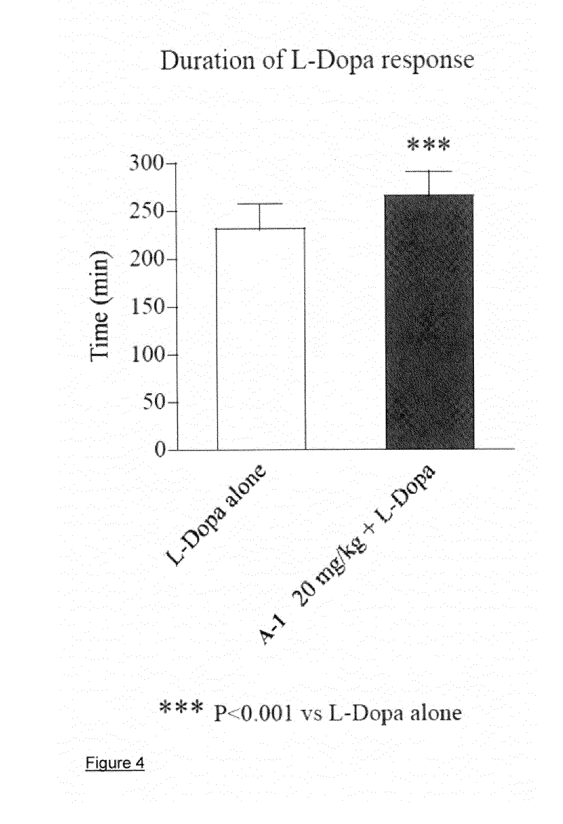

The following are further embodiments of the invention:

### EMBODIMENT 1

A nicotinic acetylcholine receptor alpha 7 activator, selected from a nicotinic acetylcholine receptor alpha 7 agonist and a nicotinic acetylcholine receptor alpha 7 positive allosteric modulator, for use in the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

### EMBODIMENT 2

A nicotinic acetylcholine receptor alpha 7 activator according to embodiment 1, wherein the movement disorder is dyskinesia associated with dopamine agonist therapy in Symptomatic Parkinsonism.

### EMBODIMENT 3

A nicotinic acetylcholine receptor alpha 7 activator according to embodiment 2, wherein the dopamine agonist is selected from levodopa; levodopa in combination with a levodopa decarboxylase inhibitor; levodopa in combination with a catechol-O-methyl transferase inhibitor; a monoamine oxidase B-inhibitor and a dopamine receptor agonist.

### EMBODIMENT 4

A nicotinic acetylcholine receptor alpha 7 activator according to embodiment 1, wherein the movement disorder is Restless Legs Syndrome.

### EMBODIMENT 5

A nicotinic acetylcholine receptor alpha 7 activator according to embodiment 1, wherein the movement disorder is Symptomatic Parkinsonism.

### EMBODIMENT 6

A nicotinic acetylcholine receptor alpha 7 activator according to embodiment 1, wherein the movement disorder is Symptomatic Parkinsonism caused by Diffuse Lewy Body Disorder.

### EMBODIMENT 7

A nicotinic acetylcholine receptor alpha 7 activator according to any of embodiments 1, 2, 3, 4, 5 or 6, wherein the nicotinic acetylcholine receptor alpha 7 activator is a nicotinic acetylcholine receptor alpha 7 agonist.

### EMBODIMENT 8

A method for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism in a subject in need of such treatment, which comprises administering to said subject a therapeutically effective amount of a nicotinic acetylcholine receptor alpha 7 activator selected from a nicotinic acetylcholine receptor alpha 7 agonist and a nicotinic acetylcholine receptor alpha 7 positive allosteric modulator.

### EMBODIMENT 9

A method according to embodiment 8, wherein the movement disorder is dyskinesia associated with dopamine agonist therapy in Symptomatic Parkinsonism.

### EMBODIMENT 10

A method according to embodiment 9, wherein the dopamine agonist is selected from levodopa; levodopa in combination with a levodopa decarboxylase inhibitor; levodopa in combination with a catechol-O-methyl transferase inhibitor a monoamine oxidase B-inhibitor and a dopamine receptor agonist.

### EMBODIMENT 11

A method according to embodiment 8, wherein the movement disorder is Restless Legs Syndrome.

### EMBODIMENT 12

A method according to embodiment 8, wherein the movement disorder is Symptomatic Parkinsonism.

### EMBODIMENT 13

A method according to embodiment 8, wherein the movement disorder is Symptomatic Parkinsonism caused by Diffuse Lewy Body Disorder.

### EMBODIMENT 14

A method according to any of embodiments 8, 9, 10, 11, 12 or 13, wherein the nicotinic acetylcholine receptor alpha 7 activator is a nicotinic acetylcholine receptor alpha 7 agonist.

### EMBODIMENT 15

A method for the treatment or delay of progression of Symptomatic Parkinsonism in a subject in need of such treatment, which comprises administering to said subject a therapeutically effective amount of (i) a dopamine agonist and (ii) a nicotinic acetylcholine receptor alpha 7 activator selected from a nicotinic acetylcholine receptor alpha 7 agonist and a nicotinic acetylcholine receptor alpha 7 positive allosteric modulator, wherein the daily dosage of the dopamine agonist is reduced compared to the daily dosage of said dopamine agonist needed to reach an equal control of Symptomatic Parkinsonism in the subject without co-administration of the nicotinic acetylcholine receptor alpha 7 activator.

1. A nicotinic acetylcholine receptor alpha 7 activator, selected from a nicotinic acetylcholine receptor alpha 7 agonist and a nicotinic acetylcholine receptor alpha 7 positive allosteric modulator, for use in the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism.

2. A nicotinic acetylcholine receptor alpha 7 activator according to claim 1, wherein the movement disorder is dyskinesia associated with dopamine agonist therapy in Symptomatic Parkinsonism.

3. A nicotinic acetylcholine receptor alpha 7 activator according to claim 2, wherein the dopamine agonist is selected from levodopa; levodopa in combination with a levodopa decarboxylase inhibitor; levodopa in combination with a catechol-O-methyl transferase inhibitor; a monoamine oxidase B-inhibitor and a dopamine receptor agonist.

4. A nicotinic acetylcholine receptor alpha 7 activator according to claim 1, wherein the movement disorder is Restless Legs Syndrome.

5. A nicotinic acetylcholine receptor alpha 7 activator according to claim 1, wherein the movement disorder is Symptomatic Parkinsonism.

6. A nicotinic acetylcholine receptor alpha 7 activator according to claim 1, wherein the movement disorder is Symptomatic Parkinsonism caused by Diffuse Lewy Body Disorder.

7. A nicotinic acetylcholine receptor alpha 7 activator according to claim 1, wherein the nicotinic acetylcholine receptor alpha 7 activator is a nicotinic acetylcholine receptor alpha 7 agonist.

8. A method for the treatment, prevention or delay of progression of a movement disorder selected from Dystonia, Dyskinesia, Chorea, Restless Legs Syndrome, Tics, Tremor, Myoclonus, Startle, Stiff Person Syndrome, Gait Disorder, Parkinson's Disease and Symptomatic Parkinsonism in a subject in need of such treatment, which comprises administering to said subject a therapeutically effective amount of a nicotinic acetylcholine receptor alpha 7 activator selected from a nicotinic acetylcholine receptor alpha 7 agonist and a nicotinic acetylcholine receptor alpha 7 positive allosteric modulator.

9. A method according to claim 8, wherein the movement disorder is dyskinesia associated with dopamine agonist therapy in Symptomatic Parkinsonism.

10. A method according to claim 9, wherein the dopamine agonist is selected from levodopa; levodopa in combination with a levodopa decarboxylase inhibitor; levodopa in combination with a catechol-O-methyl transferase inhibitor; a monoamine oxidase B-inhibitor and a dopamine receptor agonist.

11. A method according to claim 8, wherein the movement disorder is Restless Legs Syndrome.

12. A method according to claim 8, wherein the movement disorder is Symptomatic Parkinsonism.

13. A method according to claim 8, wherein the movement disorder is Symptomatic Parkinsonism caused by Diffuse Lewy Body Disorder.

14. A method according to claim 8, wherein the nicotinic acetylcholine receptor alpha 7 activator is a nicotinic acetylcholine receptor alpha 7 agonist.

15. A method for the treatment or delay of progression of Symptomatic Parkinsonism in a subject in need of such treatment, which comprises administering to said subject a therapeutically effective amount of (i) a dopamine agonist and (ii) a nicotinic acetylcholine receptor alpha 7 activator selected from a nicotinic acetylcholine receptor alpha 7 agonist and a nicotinic acetylcholine receptor alpha 7 positive allosteric modulator,
wherein the daily dosage of the dopamine agonist is reduced compared to the daily dosage of said dopamine agonist needed to reach an equal control of Symptomatic Parkinsonism in the subject without co-administration of the nicotinic acetylcholine receptor alpha 7 activator.
